(19) United States
(12) Patent Application Publication
(10) Pub. No.: US 2024/0290487 A1
(43) Pub. Date: Aug. 29, 2024

(54) SYSTEMS AND METHODS FOR USING DEEP-LEARNING ALGORITHMS TO FACILITATE DECISION MAKING IN GYNECOLOGIC PRACTICE

(71) Applicant: CORNELL UNIVERSITY, Ithaca, NY (US)

(72) Inventors: Bobak MOSADEGH, New York, NY (US); Matin TORABINIA, San Diego, CA (US); Tamatha FENSTER, Scarsdale, NY (US)

(21) Appl. No.: 18/547,725

(22) PCT Filed: Mar. 16, 2022
(86) PCT No.: PCT/US2022/020586

§ 371 Date: Aug. 24, 2023

Related U.S. Application Data
(60) Provisional application No. 63/161,884, filed on Mar. 16, 2021.

(51) Int. Cl. G16H 50/20; G16H 10/60; G16H 15/00; G16H 30/40

(52) U.S. Cl. CPC G16H 50/20; G16H 10/60; G16H 15/00; G16H 30/40

(57) ABSTRACT

Embodiments described herein provide systems and methods for improving diagnosis, screening, and treatment of patients. The disclosed systems and methods generally relate to artificial intelligence (AI) based deep learning models that can help with decision making, for example, in gynecological procedures. In various embodiments, a method of generating a model for performing gynecologic procedures is described. In various embodiments, a method of determining a success rate of a minimally invasive procedure for a patient is described. In various embodiments, a method of enhancing a diagnosis of an ovarian tumor is described. In various embodiments, a method of providing a mixed reality guidance for performing gynecological procedures is described.

### CROSS-REFERENCE TO RELATED APPLICATIONS

The present application is a National Phase of International Application No. PCT/US2022/020586, filed Mar. 16, 2022, which claims the benefit of U.S. Provisional Application No. 63/161,884, filed on Mar. 16, 2021, the contents of each of which are incorporated herein by reference as if set forth in full.

### FIELD

The embodiments disclosed herein are generally directed towards using artificial intelligence based machine learning to facilitate decision making in the diagnosis, screening, and/or treatment of patients in gynecological (gynecologic) practice.

### BACKGROUND

Gynecologic health represents an important public health concern in women, especially for particular segments of the population. For example, uterine fibroids represent the highest prevalence of benign tumors in women, with reports ranging anywhere from 4.5% to 68.6%, with a significant bias towards African American women. Furthermore, women with low socio-economic status are more likely to be referred to more invasive procedures despite their insurance coverage. It is estimated that the economic burden on the healthcare system from symptomatic women with uterine fibroids is up to $34 million. Ovarian tumors account for about 150,000 deaths in the world, providing each woman a 1 in 100 chance of dying to this disease. The survival rate at 5 years for ovarian cancer is as low as 30% and has only increased by a few percent since 1995.

Therefore, there is a need to prevent health disparity, for example by offering a better access to gynecological care (gynecologic care), such as, minimally invasive procedures, particularly for low socio-economic populations. It has been well documented that minorities are less likely to be referred for minimally invasive procedures, even though there is universal insurance coverage for them. Furthermore, women in lower socio-economic status, particularly African Americans, have been disproportionately referred for open surgery, and therefore automated tools that can provide unbiased referrals will be a significant advantage at combating this unfortunate bias.

### SUMMARY

In accordance with various embodiments, the present disclosure provides a method of generating a model for performing gynecological (gynecologic) procedures, the method comprising receiving a first dataset comprising one or more gynecological tumor features; identifying spectral and spatial features from the one or more gynecological tumor features from the first dataset; training a machine learning model using the identified spectral and spatial features, wherein the training comprises: performing a multi-class segmentation process based on the identified spectral and spatial features to produce a set of multi-class segmentation results, and classifying the identified spectral and spatial features by comparing the multi-class segmentation results with a ground-truth classification; validating the machine learning model using a second dataset; and optimizing the machine learning model by modifying the machine learning model using a third dataset.

In accordance with various embodiments, the present disclosure provides a method of determining a success rate of a minimally invasive procedure for a patient, the method comprising: receiving an imaging dataset comprising one or more scans of an anatomical area of interest for a potential procedure; analyzing the imaging dataset using a machine learning model, wherein the machine learning model is trained using a multi-class segmentation of uterine regions from a plurality of scans for a plurality of subjects; identifying one or more uterine fibroid features from the imaging dataset based on the analysis; and classifying the one or more fibroid features, individually and/or as one or more groups, based on one or more characteristics of the one or more fibroid features. In various embodiments, the method also includes determining the success rate of the minimally invasive procedure for removal of one or more uterine fibroids based on the one or more identified uterine fibroid features.

In accordance with various embodiments, the present disclosure provides a method of enhancing a diagnosis of an ovarian tumor, the method comprising: receiving an imaging dataset comprising one or more scans of the ovarian tumor; analyzing the imaging dataset using a machine learning model, wherein the machine learning model is trained using a deep learning classification and a segmentation of a plurality of scans containing benign and malignant ovarian tumors; identifying one or more ovarian tumor features from the imaging dataset based on the analysis; and determining malignancy of the ovarian tumor based on the one or more identified ovarian tumor features.

In accordance with various embodiments, the present disclosure provides a method of providing a mixed reality guidance for performing gynecological procedures, the method comprising: receiving an imaging dataset comprising scans of an anatomical area of interest; performing automated segmentation of the scans using a 3D segmentation model, wherein the 3D segmentation model is trained using a deep learning multi-class segmentation of uterine regions; extracting segmentation results comprising one or more structures of the anatomical area of interest; generating a 3D rendering using the one or more structures extracted from the automated segmentation; and displaying, via an electronic device, superimposed images from the 3D rendering overlayed with one or more scans.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 illustrates a method of generating a model for performing gynecological procedures, in accordance with various embodiments.

FIG. 2 illustrates a method of determining a success rate of a minimally invasive procedure for a patient, in accordance with various embodiments.

FIG. 3 illustrates a method of enhancing a diagnosis of an ovarian tumor, in accordance with various embodiments.

FIG. 4 illustrates a method of providing a mixed reality guidance for performing gynecological procedures, in accordance with various embodiments.

FIG. 5 is a block diagram illustrating an example computer system with which embodiments of the disclosed systems and methods, or portions thereof may be implemented, in accordance with various embodiments.

FIG. 6A shows a 3D rendering of a tumor fibroid and the same rendering within MRI cross-sections, in accordance with various embodiments.

FIG. 6B shows various images of a uterine fibroid, in accordance with various embodiments.

FIGS. 7A and 7B shows photos of augmented reality guidance systems, where FIG. 7A shows an overlaid image of muscle fibers and spheres that suggest an ideal incision point to begin myomectomy; wherein FIG. 7B shows an overlay of external wall of a uterus, uterine cavity, and location of adenomyoma to guide initial incision point, in accordance with various embodiments.

FIG. 8 illustrates a schematic of simultaneous segmentation and determination of the treatment strategy for uterine fibroids, in accordance with various embodiments.

FIG. 9 illustrates a schematic of the deep learning architecture for dual-modality network, in accordance with various embodiments.

FIG. 10 illustrates a schematic of the deep learning architecture to prognosis the recurrence of ovarian tumors, in accordance with various embodiments.

FIG. 11 shows a photo of Skills Acquisition and Innovation Laboratory (SAIL), which includes a laparoscopic trainer, in accordance with various embodiments.

FIGS. 12A and 12B illustrate a concept of rendering mixed reality (MR) guidance display, in accordance with various embodiments.

FIG. 13 shows a schematic of study design to evaluate improved performance based on mixed reality (MR) guidance, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The systems and methods disclosed herein relate to artificial intelligence (AI) based deep learning models that can improve diagnosis, screening, and treatment of patients. The use of deep learning models, in accordance with various embodiments, can improve with decision making, for example, in gynecological procedures, such that minimally invasive (MI) approaches can be performed with better outcomes and be accessible to patients from lower socio-economic populations. For example, the disclosed deep learning models can help predict a success rate of a minimally invasive procedure based on an magnetic resonance imaging (MRI) scan for fibroid removal, as described in various embodiments. Often with uterine fibroids, a major decision is determining whether fibroids can be successfully removed using a MI procedure or requires open surgery. Imaging variables in the MRI scans determine who is a candidate for MI surgery depending on the number of fibroids and exact location of myomas. Interpretation can be difficult as fibroids can lay on top of each other and present in any layer of the uterus. If patient selection is incorrect, the minimally invasive procedure can be significantly more difficult, if not impossible, thus increasing the risk of bleeding, and aborting of the MI procedure altogether. Women in lower socio-economic status, particularly African Americans, have been disproportionately referred for open surgery, and therefore automated tools that can provide unbiased referrals will be significant advantage at combating this unfortunate bias, as specified above.

The AI-based deep learning models disclosed herein can help with cancer diagnosis, screening, and treatment. Ovarian cancer, for example, is the most common form of cancer in women and has the highest rate of mortality, while uterine fibroids are the most common type of benign tumor in women. For those needing to undergo a procedure, screening is often done with ultrasound imaging due to the fact it is accessible and low-cost. However, ultrasound images provide low contrast images that are often difficult to interpret. Therefore, the use of MRI has been explored in order to provide more holistic 3D imaging of the women's reproductive organs to better determine the proper course of treatment.

For ovarian cancer, a major decision is to determine whether the tumor is benign or malignant, since this classification dictates whether women should be referred to a gynecologist or gynecologic oncologist, respectively. If a patient with a malignant tumor is mistakenly referred to a gynecologist, the patient will suffer from either, i) the inconvenience of an aborted procedure if the tumor is able to be properly identified as malignant intra-operatively, or ii) increased risk of spreading the cancer during the removal of the tumor. The greatest sensitivity and specificity of diagnosis occurs when an MRI scan is performed and interpreted by an experienced operator. Currently, patients are counseled using MRI and hand sketched representation of these MRIs. It is difficult to explain the 3D elements of the uterus to a patient and how myomas are often overlapping. It is important when taking a patient to surgery that the patient understands the degree of tumor burden and has reasonable expectations of the outcome of the fibroid surgery. The surgeon also can choose which surgical approach is most appropriate based on imaging. Some cases with high degree of tumor burden or fibroids in difficult places may be best suited for open surgery, whereas others are best served with an MIS approach. Many of these operative approach decisions will be simplified with the systems and methods discussed herein.

In accordance with various embodiments, the disclosed systems and methods can be applied intraoperatively, for example, via live real-time streaming images during fibroid surgeries. Overlaying this imaging over a uterus or floating in the operative room may allow for more efficient and safer surgery. Post operatively and pre-operative imaging can also be used to help patients and surgeons understand how the uterus has changed. Thus, the disclosed systems and methods train and apply deep learning models to automate the diagnosis and classification of ovarian tumors and uterine fibroids using radiologic features of an MRI scan, as a non-limiting example application. In addition, the disclosed systems and methods may utilize a novel mixed reality guidance system to provide a 3D rendering of, for example, uterine fibroids that can be tracked in real-time intra-operatively.

Various embodiments disclosed herein provide unique advantages over other related technologies, as described above, by providing a 3D visualization of tumor fibroids to facilitate an intuitive understanding of their shape, number, and relative positioning. This imaging will, for example, benefit preoperative surgical planning, counseling and patient education, as well as intraoperative surgical approach.

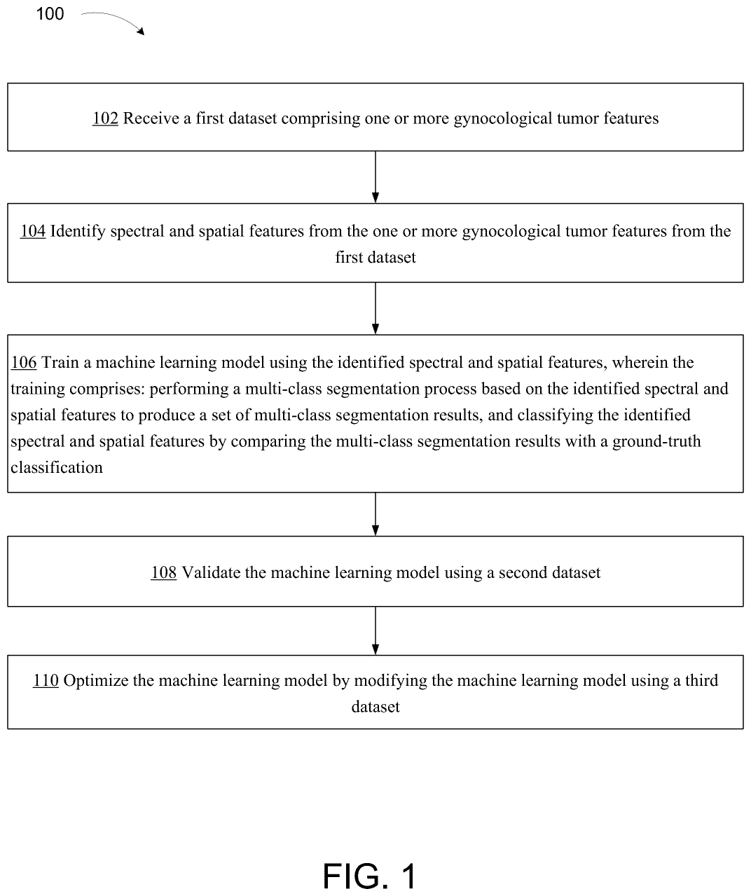

The disclosed systems and methods using artificial intelligence (AI) based deep learning models that can improve diagnosis, screening, and treatment of patients are further described with respect to the examples illustrated by FIGS. 1-13. The examples disclosed herein primarily use women's gynecological features for demonstrative purposes. However, other non-limiting examples of applicable body parts of a person, male or female, can include bladder, spine, breast, liver, pancreas, and brain. Similarly, a list of gynecological pathologies for which the disclosed systems and methods are applicable can include, but are not limited to, endometriosis, fibroids, ovarian tumors, adenomyosis, polyps, uterine septum, embryological deformities of uterus, and in various embodiments, also applicable for other obstetrics/gynecology (OB-GYN), such as, placenta location, fibroids relative to fetus, and/or fetus location, among many others.

FIG. 1 illustrates a method 100 of generating a model for performing gynecological (gynecologic) procedures, in accordance with various embodiments. The method 100 includes, at step 102, receiving a first dataset comprising one or more gynecological tumor features. In various embodiments, the first dataset can include a magnetic resonant imaging (MRI) dataset, a 3D MRI dataset, an ultrasound/sonogram dataset, a computed tomography (CT) dataset, or a doppler dataset. In various embodiments, the first dataset can include 3D MRI images of uterine fibroids and the one or more gynecological tumor features include uterine fibroid features. In various embodiments, the first dataset can include 3D MRI images of ovarian tumors and the one or more gynecological tumor features include ovarian cancer features.

As illustrated in FIG. 1, the method 100 also includes, at step 104, identifying spectral and spatial features from the one or more gynecological tumor features from the first dataset, in accordance with various embodiments. In various embodiments, the spectral and spatial features include shapes and locations of the gynecological tumor features.

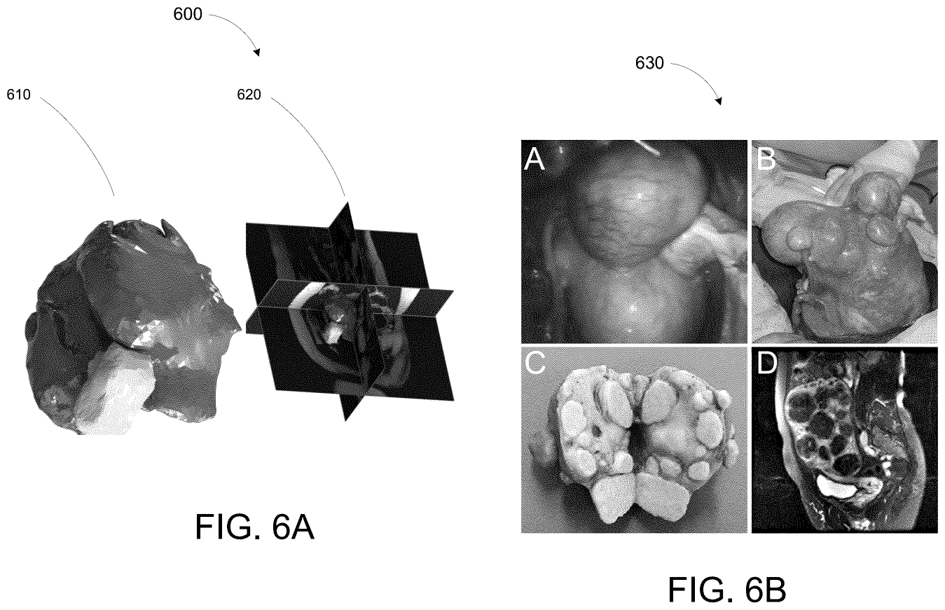

The method 100 further includes, at step 106, training a machine learning model using the identified spectral and spatial features, wherein the training can include performing a multi-class segmentation process based on the identified spectral and spatial features to produce a set of multi-class segmentation results, and classifying the identified spectral and spatial features by comparing the multi-class segmentation results with a ground-truth classification, in accordance with various embodiments. In various embodiments, the ground-truth classification includes pixel-level annotations or class-level annotations. Further details regarding the training of the machine learning model, multi-class segmentation, classifying, and ground-truth classification are described with respect to FIGS. 6-13 in the examples.

In various embodiments, performing the multi-class segmentation can include using area-based indexes to compare the multi-class segmentation results with the ground truth classification, or using distance-based indexes to further evaluate the multi-class segmentation in terms of location and shape accuracy of extracted region boundaries from the identified spectral and spatial features.

In various embodiments, classifying the spectral and spatial features can be performed via a first classifier based on gynecologic anatomies to identify a uterus, a cervix, a endometrium, or an ovary. In various embodiments, classifying the spectral and spatial features can be performed via a second classifier based on pathologies to identify as benign or malign. In various embodiments, classifying the spectral and spatial features can be performed via a third classifier based on pathologies as a fibroid, ovarian tumor, endometriosis, or adenomyosis.

In various embodiments, classifying a fibroid may include identifying and/or determining the location of fibroids, size of fibroids, positioning and number of fibroids. In various embodiments, the results of the classifying can help determine many factors, including surgical approaches, and based on symptoms the patients would have, help determine one or more treatment modalities for which the patient is eligible. In various embodiments, the machine learning model that has been trained can help determine which fibroids are benign and typical features, or which fibroids are cancerous, such as sarcoma. In various embodiments, the machine learning model is trained to produce a result with more certainty.

In various embodiments, the machine learning model can be a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

The method 100 further includes, at step 108, validating the machine learning model using a second dataset. In various embodiments, the second dataset can include a magnetic resonant imaging (MRI) dataset, a 3D MRI dataset, an ultrasound/sonogram dataset, a computed tomography (CT) dataset, or a doppler dataset. In various embodiments, the second dataset can include 3D MRI images of uterine fibroids and the one or more gynecological tumor features include uterine fibroid features. In various embodiments, the second dataset can include 3D MRI images of ovarian tumors and the one or more gynecological tumor features include ovarian cancer features.

The method 100 further includes, at step 108, optimizing the machine learning model by modifying the machine learning model using a third dataset. In various embodiments, the third dataset can include a magnetic resonant imaging (MRI) dataset, a 3D MRI dataset, an ultrasound/sonogram dataset, a computed tomography (CT) dataset, or a doppler dataset. In various embodiments, the third dataset can include 3D MRI images of uterine fibroids and the one or more gynecological tumor features include uterine fibroid features. In various embodiments, the third dataset can include 3D MRI images of ovarian tumors and the one or more gynecological tumor features include ovarian cancer features.

In various embodiments, the first, second, and third datasets comprise a magnetic resonant imaging (MRI) dataset, a 3D MRI dataset, an ultrasound/sonogram dataset, a computed tomography (CT) dataset, or a doppler dataset, and subjects' metadata. In various embodiments, the subjects' metadata (e.g., structured tabular data as described herein) include one or more of the pre-operative, procedural, and/or post-operative attributes as follows: i) pre-operative: social history (age, BMI, surgical history, ADL, etc.), socioeconomics (occupation, marital status, health maintenance-pap, vaccines, etc.), imaging (IOTA score), and blood work (CA-125, HE4, ROMA test, OVA-1); ii) procedural: anesthesia ASA, estimated blood Loss, total IV fluids, operative time, year of resident, robot used, laparoscope used, conversion rate, etc.; and/or iii) post-operative: pathology, ovarian tumor size, fibroid weight, ovarian tumor size.

In accordance with various embodiments and as discussed above, furthermore, the systems and methods disclosed herein utilize one or more AI-based deep learning models to improve diagnosis, screening, and treatment of patients. In various embodiments, the process flow can also be as follows: an AI model is used in a hierarchical fashion to perform both increasingly more sophisticated diagnosis and prognosis. Initially, the images, such as those described herein with respect to first, second, and/or third dataset, that include MRI, CT, ultrasound, doppler, etc., can be processed to classify the image as normal or pathologic using retrospective images (e.g., those annotated by an expert) designated as such for a variety of aforementioned diseases. If the image is designated as normal, basic gynecologic structures can be segmented (e.g., uterus, endometrium, ovaries, etc.) along with one or more reference organs (e.g., bladder, spine, breast, liver, pancreas, brain, etc., including the list of body parts as disclosed herein) using ground-truth annotations from retrospective normal scans. If the image is designated as pathologic, the type of pathology can be classified (e.g., fibroids, ovarian cancer, endometriosis, etc., including the list of gynecological pathologies as disclosed herein).

For a specific pathology, segmentations based on retrospective ground-truth annotations are trained. In addition, specific quantitative metrics and classifications can be outputted, in various embodiments. For example, uterine fibroids have specific annotations for the fibroids, distorted uterine wall, and distorted endometrium, among many others. In various embodiments, quantitative metrics include the number of fibroids, size of each fibroid, submucosal and subserosal distances. Classifications will be fibroid layer location (e.g., subserosal, intramural, submucosal, pedunculated, etc.) and position (anterior, posterior, left body, right body, fundus, cervical, etc.).

For each type of pathology, imaging with and without tabular data can be used to determine the success rate of each type of known procedure, providing a scalar value for the confidence the model has that the specific procedure will be successful, in accordance with various embodiments.

In various embodiments, manual classifications can be done by a physician, such that only downstream models are utilized. In such cases, if a patient is already diagnosed with uterine fibroids, the models can be used to just perform for that specific pathology. In various embodiments, the disclosed model structure is a hierarchical model that includes diagnosis/prognosis and is modular based on the disease type.

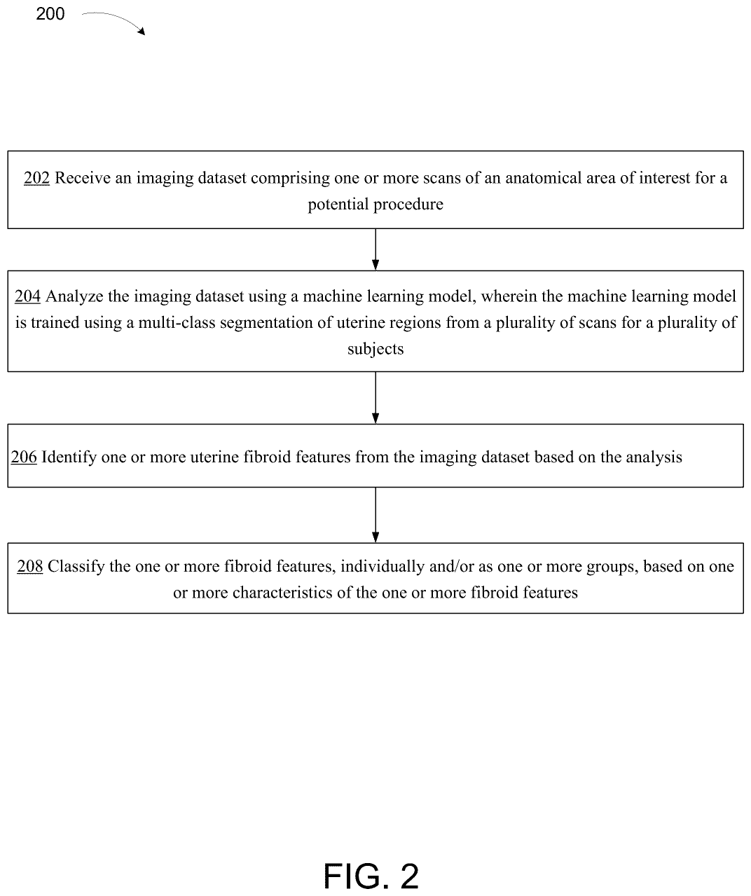

FIG. 2 illustrates a method 200 of determining a success rate of a minimally invasive procedure for a patient, in accordance with various embodiments. The method 200 includes, at step 202, receiving an imaging dataset comprising one or more scans of an anatomical area of interest for a potential procedure. In various embodiments, the imaging dataset can include a magnetic resonant imaging (MRI) dataset, a 3D MRI dataset, an ultrasound/sonogram dataset, a computed tomography (CT) dataset, or a doppler dataset. In various embodiments, the imaging dataset can include 3D MRI images of uterine fibroids and the one or more gynecological tumor features include uterine fibroid features, such as a list of the features as described herein.

The method 200 also includes, at step 204, analyzing the imaging dataset using a machine learning model, wherein the machine learning model is trained using a multi-class segmentation of uterine regions from a plurality of scans for a plurality of subjects. In various embodiments, the training of the machine learning model can include performing a multi-class segmentation process based on a plurality of uterine fibroid features identified in a training dataset to produce a set of multi-class segmentation results, and classifying the plurality of uterine fibroid features by comparing the multi-class segmentation results with a ground-truth classification. In various embodiments, the ground-truth classification includes pixel-level annotations or class-level annotations. Further details regarding the training of the machine learning model, multi-class segmentation, classifying, and ground-truth classification are described with respect to FIGS. 6-13 in the examples.

In various embodiments, performing the multi-class segmentation of the method 200 can include using area-based indexes to compare the multi-class segmentation results with the ground truth classification, or using distance-based indexes to further evaluate the multi-class segmentation in terms of location and shape accuracy of extracted region boundaries from the identified spectral and spatial features.

In various embodiments, classifying the spectral and spatial features can be performed via a first classifier based on gynecologic anatomies to identify a uterus, a cervix, a endometrium, or an ovary. In various embodiments, classifying the spectral and spatial features can be performed via a second classifier based on pathologies to identify as benign or malign. In various embodiments, classifying the spectral and spatial features can be performed via a third classifier based on pathologies as a fibroid, ovarian tumor, endometriosis, or adenomyosis.

In various embodiments, classifying a fibroid may include identifying and/or determining the location of fibroids, size of fibroids, positioning and number of fibroids. In various embodiments, the results of the classifying can help determine many factors, including surgical approaches, and based on symptoms the patients would have, help determine one or more treatment modalities for which the patient is eligible. In various embodiments, the machine learning model that has been trained can help determine which fibroids are benign and typical features, or which fibroids are cancerous, such as sarcoma. In various embodiments, the machine learning model is trained to produce a result with more certainty.

In various embodiments, the machine learning model can include a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

In various embodiments, the deep learning model is a dual-modality multitask deep learning model trained using the plurality of 3D volumetric MRI scans and patient-level metadata, wherein the CNN is trained using the plurality of 3D volumetric MRI scans, and wherein the patient-level metadata is encoded as a feature vector.

The method 200 also includes, at step 206, identifying one or more uterine fibroid features from the imaging dataset based on the analysis. In various embodiments, the one or more identified uterine fibroid features comprise a shape, a size, a number of, and/or relative positioning of the one or more uterine fibroids in the anatomical area of interest.

The method 200 also includes, at step 208, classify the one or more fibroid features, individually and/or as one or more groups, based on one or more characteristics of the one or more fibroid features.

The method 200 may include outputting, via an output device, one or more representations of the one or more characteristics of the one or more fibroid features, wherein the one or more characteristics of the one or more fibroid features comprises a success rate of one or more types of surgical intervention for the one or more fibroid features.

The method 200 may include outputting, via an output device, one or more representations of the one or more fibroid features, either in isolation or in combination with the one or more characteristics of the one or more fibroid features.

In various embodiments, the one or more characteristics of the one or more fibroid features used in the act of classifying the one or more fibroid features can comprise a fibroid shape, a fibroid size, a number of fibroids, a fibroid position relative to at least one anatomical structure, a fibroid position relative to a blood vessel, or a fibroid position relative to at least one other fibroid.

The method 200 may include determining the success rate of the minimally invasive procedure for removal of one or more uterine fibroids based on the one or more identified uterine fibroid features. In various embodiments, the success rate of the minimally invasive procedure hinges on whether one or more target fibroids are actually removed, whether a serious complication can arise from the MI procedure, whether the likelihood of the procedure being aborted, and/or whether the likelihood of the procedure being converted to an open surgery. In various embodiments, the minimally invasive procedure includes robotic or laparoscopic surgery (as opposed to open surgery). In various embodiments, the minimally invasive procedures for fibroid removal may include, but not limited to, uterine artery embolization, hysteroscopic myomectomy, radiofrequency ablation (e.g., Acessa, Sonata), and magnetic resonance-guided focused ultrasound surgery (MRgFUS).

In accordance with various embodiments, the success rate of the minimally invasive procedure is determined to be low when the one or more uterine fibroids are identified to be difficult to remove. In accordance with various embodiments, the success rate of the minimally invasive procedure is determined to be high when the one or more uterine fibroids are identified to be easy to remove without an increased risk of bleeding or an increased length of the potential surgery.

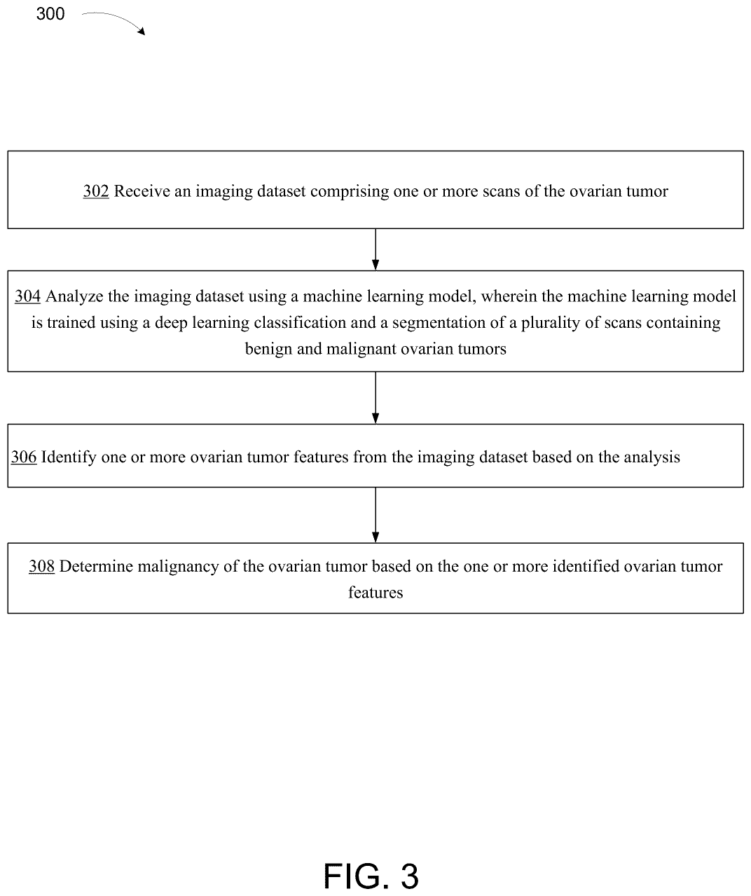

FIG. 3 illustrates a method 300 of enhancing a diagnosis of an ovarian tumor, in accordance with various embodiments. The method 300 includes, at step 302, receiving an imaging dataset comprising one or more scans of the ovarian tumor. In various embodiments, the imaging dataset can include a magnetic resonant imaging (MRI) dataset, a 3D MRI dataset, an ultrasound/sonogram dataset, a computed tomography (CT) dataset, or a doppler dataset. In various embodiments, the first dataset can include 3D MRI images of ovarian tumors and the one or more gynecological tumor features include ovarian cancer features as described herein.

The method 300 includes, at step 304, analyzing the imaging dataset using a machine learning model, wherein the machine learning model is trained using a deep learning classification and a segmentation of a plurality of scans containing benign and malignant ovarian tumors. In various embodiments, the training of the machine learning model can include performing the deep learning classification and segmentation based on a plurality of ovarian tumor features identified in a training dataset to produce a set of multi-class segmentation results, and classifying the plurality of ovarian tumor features by comparing the multi-class segmentation results with a ground-truth classification. In various embodiments, the ground-truth classification includes pixel-level annotations or class-level annotations. Further details regarding the training of the machine learning model, multi-class segmentation, classifying, and ground-truth classification are described with respect to FIGS. 6-13 in the examples.

In various embodiments, classifying the spectral and spatial features can be performed via a first classifier based on gynecologic anatomies to identify a uterus, a cervix, a endometrium, or an ovary. In various embodiments, classifying the spectral and spatial features can be performed via a second classifier based on pathologies to identify as benign or malign. In various embodiments, classifying the spectral and spatial features can be performed via a third classifier based on pathologies as a fibroid, ovarian tumor, endometriosis, or adenomyosis.

In various embodiments, classifying a tumor may include identifying and/or determining the location of tumor, size of tumors, positioning and number of tumors. In various embodiments, the results of the classifying can help determine many factors, including surgical approaches, and based on symptoms the patients would have, help determine one or more treatment modalities for which the patient is eligible. In various embodiments, the machine learning model that has been trained can help determine which fibroids are benign and typical features, or which fibroids are cancerous, such as sarcoma. In various embodiments, the machine learning model is trained to produce a result with more certainty.

In various embodiments, the machine learning model can include a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

In various embodiments, the deep learning model is a dual-modality multitask deep learning model trained using the plurality of 3D volumetric MRI scans and patient-level metadata, wherein the HIFUNet is trained using multi-class segmentation of an ovarian tumor, designating two diagnostic categories as benign or malignant, and wherein the CNN is trained using an ovarian tumor segmentation.

The method 300 includes, at step 306, identifying one or more ovarian tumor features from the imaging dataset based on the analysis. In various embodiments, the one or more identified ovarian tumor features may include a shape, a size, a number of, and relative positioning of one or more ovarian tumors in the scans. In various embodiments, the one or more identified ovarian tumor features may include intensity values, patterns in intensity, e.g., layers, gradients, internal structures, etc.

The method 300 includes, at step 308, determining malignancy of the ovarian tumor based on the one or more identified ovarian tumor features. In various embodiments, the determining of the malignancy depends on multifactorial based on the trained weights of the learning model used.

In various embodiments, the method 300 can include outputting, via an output device, one or more representations of the one or more ovarian tumor features or one or more representations of a success rate of one or more types of surgical intervention for the one or more ovarian tumor features.

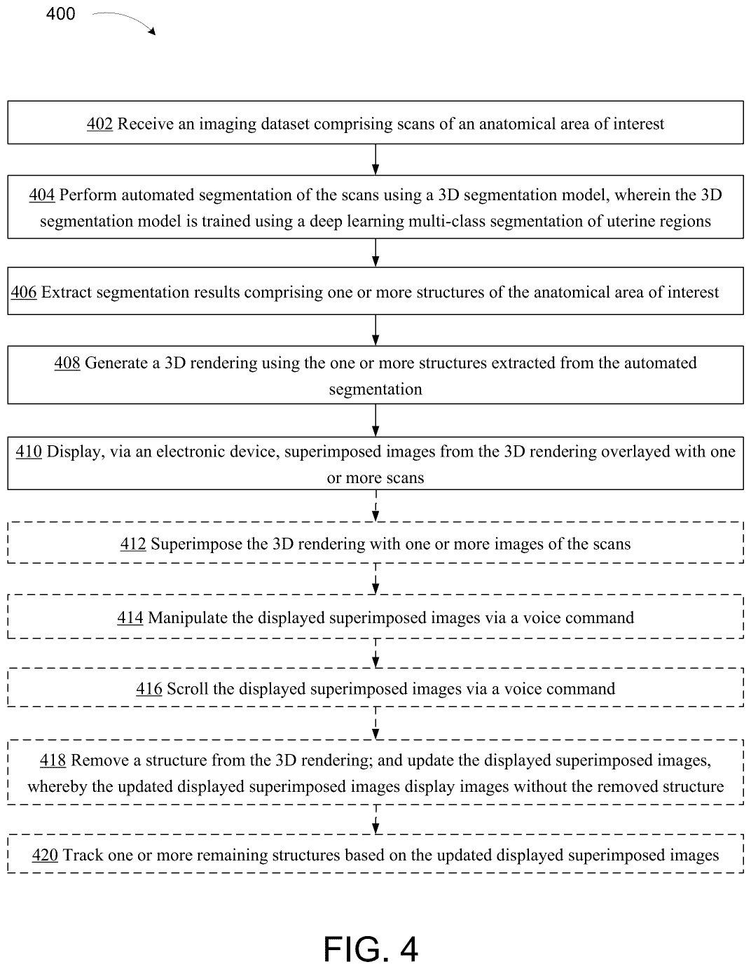

FIG. 4 illustrates a method 400 of providing a mixed reality guidance for performing gynecological procedures, in accordance with various embodiments. The method 400 includes, at step 402, receiving an imaging dataset comprising scans of an anatomical area of interest. In various embodiments, the imaging dataset can include a magnetic resonant imaging (MRI) dataset, a 3D MRI dataset, an ultrasound/sonogram dataset, a computed tomography (CT) dataset, or a doppler dataset. In various embodiments, the imaging dataset can include images from any of the datasets disclosed herein of uterine fibroids and the one or more gynecological tumor features include uterine fibroid features. In various embodiments, the imaging dataset can include images from any of the datasets disclosed herein of ovarian tumors and the one or more gynecological tumor features include ovarian cancer features.

The method 400 includes, at step 404, performing automated segmentation of the MRI scans using a 3D segmentation model, wherein the 3D segmentation model is trained using a deep learning multi-class segmentation of uterine regions. In various embodiments, the training of the 3D segmentation model can include performing a multi-class segmentation process based on the identified spectral and spatial features to produce a set of multi-class segmentation results, and classifying the identified spectral and spatial features by comparing the multi-class segmentation results with a ground-truth classification, in accordance with various embodiments. In various embodiments, the ground-truth classification includes pixel-level annotations or class-level annotations. Further details regarding the training of the machine learning model, multi-class segmentation, classifying, and ground-truth classification are described with respect to FIGS. 6-13 in the examples.

In various embodiments, performing the multi-class segmentation can include using area-based indexes to compare the multi-class segmentation results with the ground truth classification, or using distance-based indexes to further evaluate the multi-class segmentation in terms of location and shape accuracy of extracted region boundaries from the identified spectral and spatial features.

In various embodiments, classifying the spectral and spatial features can be performed via a first classifier based on gynecologic anatomies to identify a uterus, a cervix, a endometrium, or an ovary. In various embodiments, classifying a fibroid may include identifying and/or determining the location of fibroids, size of fibroids, positioning and number of fibroids. In various embodiments, the results of the classifying can help determine many factors, including surgical approaches, and based on symptoms the patients would have, help determine one or more treatment modalities for which the patient is eligible. In various embodiments, the machine learning model that has been trained can help determine which fibroids are benign and typical features, or which fibroids are cancerous, such as sarcoma. In various embodiments, the machine learning model is trained to produce a result with more certainty.

In various embodiments, the machine learning model can be a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

The method 400 includes, at step 406, extracting segmentation results comprising one or more structures of the anatomical area of interest, including, for example, but not limited to a uterus, a fibroid, a cervix, a endometrium, a bladder, or an ovary.

The method 400 includes, at step 408, generating a 3D rendering using the one or more structures extracted from the automated segmentation.

The method 400 includes, at step 410, displaying, via an electronic device, superimposed images from the 3D rendering overlayed with one or more scans. In various embodiments, the electronic device may include a display, a monitor, a mixed reality device, an artificial reality device, or a virtual reality device.

In various embodiments, the method 400 can optionally include, at step 412, superimposing the 3D rendering with one or more images of the scans. In various embodiments, the 3D rendering are segmented from the images, such as MRI images, which are co-registered since they use the same coordinate system during reconstruction of the 3D rendering from the MRI image stack. Additional details are described herein.

In various embodiments, the method 400 can optionally include, at step 414, manipulating the displayed superimposed images via a voice command. In various embodiments, the method 400 can optionally include, at step 416, scrolling the displayed superimposed images via a voice command. In various embodiments, the method 400 can optionally include, at step 418, removing a structure from the 3D rendering and updating the displayed superimposed images, whereby the updated displayed superimposed images display images without the removed structure. In various embodiments, the method 400 can optionally include, at step 420, tracking one or more remaining structures based on the updated displayed superimposed images.

In various embodiments, a system using the mixed reality guidance based on the aforementioned method 400 can be an end-to-end software that can allow a physician to upload a MRI scan and have it automatically render in 3D the fibroids along with surrounding anatomic landmarks, such as the uterine wall, endometrium, and bladder. The 3D rendering can be viewed in a conventional 2D display or within a 3D headset (i.e., virtual reality, augmented reality, mixed reality). The software can have three viewing modes: i) pre-procedural, which can visualize the scan and allow for path planning to be discussed with other physicians and the patient, ii) intra-procedural, which can allow the tracking of fibroid removal and guidance for the order of each fibroids removal, and iii) post-procedural, which can allow for specific notes to be annotated with voice-commands and the steps of the procedure to be recorded and replayed as a movie.

An example process flow for the 3D rendering can be as follows: Step 1—upload MRI scan, Step 2—view/plan pre-procedurally, Step 3—trace steps intra-procedurally, and Step 4—analyze steps post-procedurally. Although the 3D rendering can be focused on uterine fibroids, the algorithms developed can be applied to any gynecological procedure in which MRI scans are taken prior to the procedure, and thus has the potential for significant impact in many aspects of both women's healthcare. The deep-learning based 3D rendering can be applied to ovarian tumors. For ovarian cancer, a major decision is to determine whether the tumor is benign or malignant, since this classification dictates whether women should be referred to a gynecologist or gynecologic oncologist, respectively.

In various embodiments, the systems and methods for the various embodiments discussed herein can be implemented via computer software or hardware via a computer system as discussed below.

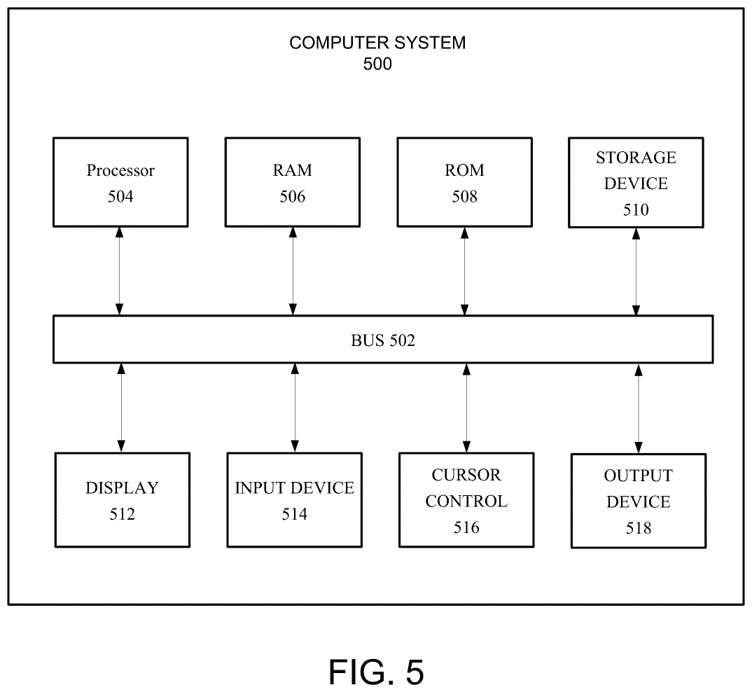

FIG. 5 is a block diagram illustrating an example computer system 500 with which embodiments of the disclosed systems and methods, or portions thereof may be implemented, in accordance with various embodiments. For example, the illustrated computer system can be a local or remote computer system operatively connected to a control system for controlling or monitoring the systems and methods of the various embodiments herein. In various embodiments of the present teachings, computer system 500 can include a bus 502 or other communication mechanism for communicating information and a processor 504 coupled with bus 502 for processing information. In various embodiments, computer system 500 can also include a memory, which can be a random-access memory (RAM) 506 or other dynamic storage device, coupled to bus 502 for determining instructions to be executed by processor 504. Memory can also be used for storing temporary variables or other intermediate information during execution of instructions to be executed by processor 504. In various embodiments, computer system 500 can further include a read only memory (ROM) 508 or other static storage device coupled to bus 502 for storing static information and instructions for processor 504. A storage device 510, such as a magnetic disk or optical disk, can be provided and coupled to bus 502 for storing information and instructions.

In various embodiments, computer system 500 can be coupled via bus 502 to a display 512, such as a cathode ray tube (CRT) or liquid crystal display (LCD), for displaying information to a computer user. An input device 514, including alphanumeric and other keys, can be coupled to bus 502 for communication of information and command selections to processor 504. Another type of user input device is a cursor control 516, such as a mouse, a trackball or cursor direction keys for communicating direction information and command selections to processor 504 and for controlling cursor movement on display 512. This input device 514 typically has two degrees of freedom in two axes, a first axis (i.e., x) and a second axis (i.e., y), that allows the device to specify positions in a plane. However, it should be understood that input devices 514 allowing for 3-dimensional (x, y and z) cursor movement are also contemplated herein. In accordance with various embodiments, components 512/514/516, together or individually, can make up a control system that connects the remaining components of the computer system to the systems herein and methods conducted on such systems, and controls execution of the methods and operation of the associated system.

In various embodiments, the computer system 500 includes an output device 518. In various embodiments, the output device 518 can be a wireless device, a computing device, a portable computing device, a communication device, a printer, a graphical user interface (GUI), a gaming controller, a joy-stick controller, an external display, a monitor, a mixed reality device, an artificial reality device, or a virtual reality device.

Consistent with certain implementations of the present teachings, results can be provided by computer system 500 in response to processor 504 executing one or more sequences of one or more instructions contained in memory 506. Such instructions can be read into memory 506 from another computer-readable medium or computer-readable storage medium, such as storage device 510. Execution of the sequences of instructions contained in memory 506 can cause processor 504 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 504 for execution. Such a medium can take many forms, including but not limited to, non-volatile media, volatile media, and transmission media. Examples of non-volatile media can include, but are not limited to, dynamic memory, such as memory 506. Examples of transmission media can include, but are not limited to, coaxial cables, copper wire, and fiber optics, including the wires that comprise bus 502.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 504 of computer system 500 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 500 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 500, whereby processor 504 would execute the analyses and determinations provided by these engines, subject to instructions provided by any one of, or a combination of, memory components 506/508/510 and user input provided via input device 514.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

In accordance with various embodiments, an example method for visualizing tumor fibroids can also be provided that can comprise i) generating a library of MRI scans from patients with tumor fibroids, ii) manually segmenting those scans to serve as ground truth images of the fibroid structures within the scan, iii) automating the segmentation of a tumor fibroid in a MRI scan using a deep learning model, and iv) displaying the 3D volume of the tumor fibroid in a CAD software for 2D display, or in a virtual, augmented, or mixed reality headset for 3D display. Features in the software can allow the fibroids to be viewed within cross-sections of the MRI to give context of surrounding anatomic structures.

In accordance with various embodiments, an example system utilizing the various methods can include a server computer that can accept an uploaded MRI, and then the machine learning algorithm automatically processes the scan. A convolutional neural network (e.g. U-Net) could be used as a deep learning methods to do the image segmentation. A 3D rendering file could also be generated automatically that could be downloaded in a headset for visualization. FIG. 6A shows a 3D rendering 600 of a tumor fibroid 610 (left) and the same rendering within MRI cross-sections 620 (right), in accordance with various embodiments. For comparison, FIG. 6B shows various images 630 (e.g., A, B, C, and D) of a uterine fibroid, in accordance with various embodiments. The various embodiments herein can be used for various purposes including patient education, pre-procedural planning, and intra-procedural guidance. In various embodiments, the 3D rendering and/or the various systems and methods disclosed herein can be used as a supplementary additive tool to guide referrals and surgical approaches. Further details of the disclosed systems and methods are described via the examples as set forth below.

### Examples

The content hereby incorporated into the disclosure and are provided as illustrative examples of the disclosure, not as restrictive descriptions. It is submitted with the understanding that they will not be used to limit the scope or meaning of the claims. It can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

In accordance with various embodiments, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

Artificial Intelligence (AI) algorithms hold a unique position in revolutionizing healthcare systems, from image analysis and information retrieval to forecasting and decision making. Three different methods, including logistic regression, Artificial Neural Networks (ANNs), and Classification Regression Trees (CARTs) can be used to compare endometrial cancer's diagnostic accuracy in postmenopausal women presenting with vaginal bleeding. ANN can outperform the CART and logistic regression model, showing higher accuracy, sensitivity, and specificity. A Neural Network (NN) model can be used to identify adnexal masses in ultrasound images. The NN's better performance over less experienced examiners and Shallow Learning (SL) such as Support Vector Machine (SVM). For example, six different AI models: the probabilistic neural network (PNN), gene expression programming classifier, k-Means algorithm, Multilayer Perceptron Network (MLP), radial basis neural function network, and SVM are described. The models' performance can be compared based on the accuracy, sensitivity, specificity, and area under the receiver operating characteristic curve (AUC). The study found the PNN model appreciably more effective in predicting patients' survival rate. Other researchers drew a similar conclusion that the Deep Learning (DL) models (i.e., ANN, PNN) outperform SL models (i.e., CARTs, SVM) in the gynecologic oncology realm.

In contrast to an ANN model, the neurons in a CNN model are not connected to all other neurons yet connected only to a small region of neurons in the previous layer, leveraging the features learned in the previous layers. Furthermore, ANNs are unsatisfactory for image datatype because these networks lead to over-fitting quickly due to the images' size. The advancement of deep learning architecture like CNN and deep autoencoders not only transforms typical computer vision tasks like object detection but are also efficient in other related tasks like classification, localization, tracking, and image segmentation. A state-of-the-art U-Net can be implemented by replacing the pooling operators in Fully Convolutional Network (FCN) with upsampling operators, allowing the input image's resolution retention. U-Net's performance in segmenting medical images, notably with a small training dataset, promises the potential of such Encoder-Decoder architecture. The U-Net can be extended for processing other medical images including but not limited to the Xenopus kidney and MRI volume segmentation of prostate, retinal vessels, liver and tumors in CT scans, ischemic stroke lesion, intervertebral disc and pancreas. Nevertheless, the uncertainty of the location, the numbers, and the sizes of uterine fibroids result in an increase of complexity for segmentation and failure to employ feature learning from different levels efficiently.

Mixed reality holds the promise of providing digital enhancement based on pre-procedural images and planning. In addition, the ability to provide overlaid images that have depth perception is a significant improvement over traditional 3D models that are displayed on 2D screens. Despite these advantages, there has been little adoption of mixed reality into clinical interventions due to limitations in hardware, lack of streamlined methods to generate 3D data to render in the mixed reality environment, and an inability to provide real-time updates to the model due to events occurring during the procedure. With the recent advancements in hardware (e.g., Microsoft HoloLens), and new techniques in machine learning, fully interactive methods can be created to guide procedures based on pre-operative and intra-operative imaging. Furthermore, 3D printed models of anatomic structures are becoming more prevalent for use in visualizing patient anatomy and enabling mock procedures to be performed for practicing and planning.

The following is an example for determining if a deep learning model can predict the success rate of a minimally invasive procedure based on an MRI scan for fibroid removal. A new dual-modality multitask deep learning model can be developed to jointly predict the prognosis (regression task) for the removal of uterine fibroids using a minimally invasive laparoscopic approach. Both 3D volumetric MRI images and patients' metadata (structured tabular data) can be used to train the deep learning architecture, e.g., disclosed methods and systems described herein. The model can be trained with retrospective images with ground truth classifications determined by procedure conversion to open surgery due to an inability to complete the procedure laparoscopically. The development of this new deep learning model can ensure that patients with uterine fibroids can be referred to the appropriate type of procedure to avoid having their cases aborted and/or suffering unnecessary adverse outcomes.

The following is an example for developing a deep learning model to predict which patients are successful candidates for a minimally invasive procedure based on malignancy of an ovarian tumor. A new dual-modality multitask deep learning model can be developed to jointly classify a tumor as malignant or benign based on radiologic markers from a set of retrospective MRI scans. Ground-truth classification can be determined by biologic tumor markers obtained post-procedurally. The development of this model is the basis to automate the referral of patients to either a gynecologist or oncology gynecologist for the removal of ovarian tumors using laparoscopy.

The following is an example for determining if mixed reality guidance provides improved procedural outcomes. A deep learning model is developed to automate the segmentation of uterine fibroids from a set of retrospective MRI scans. A mixed reality environment rendered from these patient-specific segmentations can enable physicians to visualize the relative positions of tumor fibroids for pre- and intra-procedural planning. The custom-software can allow voice-activated tracking of uterine fibroid removal in real-time. Intra-procedural guidance with and without using the mixed reality display can be compared in a pre-clinical study using a 3D printed model. The study can consist of physicians with and without experience performing laparoscopic procedures. The development of this guidance tool can potentially lower the learning curve for these minimally invasive procedures such that these lower risk procedures can be utilized for lower socio-economic populations.

For gynecologic procedures, these examples can provide specific guidance and training for the localization of myomas during laparoscopy that would otherwise be very challenging due to minor changes in the surface of the uterus (i.e., types 2-4 of the International Federation of Gynecology and Obstetrics classification system). Furthermore, fibroids could be present in multiple locations, and not always easily localized.

Of particular interest are small fibroids (e.g., <1 cm), which are more likely to be left in place after laparoscopy (because there is no tactile feedback) compared with laparotomy. In addition, recurrence after laparoscopic myomectomy has been described as more likely than after myomectomy with the use of laparotomy. In contrast, robotic myomectomy requires more technical improvement, because the residual fibroid volume has described to be as much as five times greater than after laparotomy, and the recurrence rate 5 years after laparoscopic myomectomy reaches more than 50% in many series reported in the literature.

Currently, the method to localize fibroids seems to be inadequate for small and/or multiple fibroids. A mixed reality guidance system, in accordance with various embodiments, can facilitate visualizing the positions of these fibroids, as seen in previously reported work.

Since the early efforts to apply AI models in gynecologic oncology, advancements have been made to the algorithms and methodologies. It is clear that the deep learning architectures have shed light upon the success of their performance and given us an improved understanding of their capability. Despite the existing deep learning architectures, there is paucity of literature on leveraging Convolutional Neural Networks and deep autoencoders in gynecologic practice, particularly for ovarian cancer staging, diagnostic and treatment planning of uterine fibroids.

Very few contributions have been reported for segmenting uterus and uterine fibroids from MRI. Ben-Zadok et al. presented an expert-supervised segmentation method where the physicians had to provide feedback regarding the first phase's automatic segmentation results. The study found that an experienced physician's oversight is inevitable to achieve segmentation within acceptable accuracy. Militello et al. reported a region-growing segmentation approach for uterine fibroids. The proposed method required dataset interpolation, pre-processing filtering, selection of seed-point, and post-processing filtering. A few others reported other mixed methods such as Fuzzy C-Means (FCM) and the active shape model (ASM) to segment the fibroid area. However, these conventional and reported automatic methods are prone to several limitations in pre- and post-processing filtering and physician interventions. Zhang et al. recently proposed a Global Convolutional Network (GCN) with Deep Multiple Atrous Convolutions (DMAC) to automatically segment the uterus, uterine fibroids, and spine. The results were compared with conventional and other deep learning methods and demonstrated a significant improvement in accuracy (up by 8%) and robustness compared to state-of-the-art segmentation methods. Despite the promising result, the author reported boundary inaccuracies in patients depicting multiple fibroids.

Notwithstanding the aforementioned studies and a variety of employed techniques, there remains a scarcity of objective evidence reasoning a proper treatment plan for uterine fibroid removal (laparoscopy vs. laparotomy). Furthermore, the uncertainty in shape and the number of fibroids, the unclear boundaries, and the presence at different uterus regions highlight the necessity of a robust model incorporating all the factors.

The disclosed methods and systems relate to deep learning-assisted gynecological framework that can allow healthcare organizations flexibility and scalability to ensure that patients with fibroids can be referred appropriately to the best treatment based on anatomy, without social bias. Notably, in the case of ovarian tumors, an End-to-End automated classification system, as disclosed herein, can be one tool to regulate patients' referral to either a gynecologist or oncology gynecologist for the removal of ovarian tumors using laparoscopy. Furthermore, the developed guidance methods and benchtop models can be used for pre-procedural planning and practice and as a teaching tool for residents and fellows. This method can be applied to any transcatheter procedure in which a MRI scan is taken prior to the procedure, and thus has the potential for significant impact. The mixed reality-based virtual coach system can work with any imaging system (e.g., GE, Philips, Siemens C-arm), thus allowing for easy adoption by clinics. Since the developed technology is open-source, it can enable a method for sharing training procedures for clinicians to share and standardized methodologies for specific procedures.

The disclosed systems and methods can be applied to existing architecture for ovarian tumors and uterine fibroids. 3D MRI datasets (ovarian tumors, and uterine fibroids datasets) can be trained using, for example, Deep Medic, a famous work which won the ISLES 2015 competition and became the state-of-the-art performance on 3D volumetric brain scans. Besides being a 3D CNN, another fact that makes the Deep Medic a rational architecture candidate to be tested on fibroids dataset is its capability in the face of variations in brain lesion size across different scans, causing imbalances in training samples. Yet, Deep Medic has always been implemented for brain tumor segmentation, and this would allow us to determine the robustness of the network in the presence of other organs using MRI scans. Moreover, the Y-Net architecture, which was introduced in 2018 as a joint segmentation and classification network to diagnose breast biopsy images, can be leveraged to implement the disclosed systems and methods. Y-Net outperformed the plain and residual encoder-decoder networks by 7% and 6%, respectively.

For the development of new deep learning model architectures, the new and/or novel architectures of deep learning can be used to obtain better performance in segmentation, classification and prediction of gynecologic procedures.

In various embodiments, a novel 3D segmentation approach of fibroids can include using HIFUNet. The state-of-the-art Encoder-Decoder global convolutional network (a.k.a. HIFUNet), which was published in late 2020, outperformed other deep learning models (i.e., U-Net, HRNet, and CE-Net) during the segmentation of uterus and uterine fibroids. However, one of the significant shortcomings of this work is boundary inaccuracies in patients depicting multiple fibroids, which can often occur. Such limitation can be mitigated by employing 3D CNN into HIFUNet, granting 3D segmentation of MRI scans. 3D CNNs can consider both 1D and 2D CNNs by simultaneously extracting spectral and spatial features from the input volume instead of 2D CNN (only spatial) or ID CNN (only spectral). In doing so, 3D CNNs can be implemented into HIFUNet.

In various embodiments, a novel joint segmentation and classification framework can be implemented for gynecologic practice. The existing Y-Net's encoding blocks can be replaced with the encoder module ResNet101 and the feature extractor GCN, which both are part of the HIFUNet. Such expansion of the network not only allows taking advantage of GCN with deep multiple convolutions, which showed promising results in, but also enables the model to perform segmentation and classification jointly. The disclosed joint deep learning model ensures the correct treatment strategy for both patients diagnosed with ovarian tumors and uterine fibroids. In doing so, a joint Deep-learning assisted treatment planning can be implemented in gynecologic practice.

A new dual-modality multitask deep learning model is described. While imaging variables (i.e., shape, number, and location of fibroids) are rich information aiding the selection of the correct treatment planning for patients with uterine fibroids, the performance of the deep learning model can be improved by incorporating both 3D volumetric MRI images and patient-level metadata (structured tabular data). To this end, the Convent module (containing the encoder module ResNet101 and the feature extractor GCN) are employed to be receptive to MRI images and implement a Dense module for the encoded patient-level metadata. In doing so, a new dual-modality multitask deep learning architecture can be developed in gynecologic practice.

The development of a 3D printed training model for removal of uterine fibroids is described. The training model system can be fabricated using a digital 3D printer, which uses Polyjet technology to allow several materials of varying stiffness to be printed within a single model to distinguish fibroids from surrounding tissue. Integrated channels can also recapitulate major arteries and provide a visual cue if damaged. Furthermore, this training phantom can be designed from patient-specific segmentations that provide a workflow, such that these models can be used for pre-procedural planning for future cases.

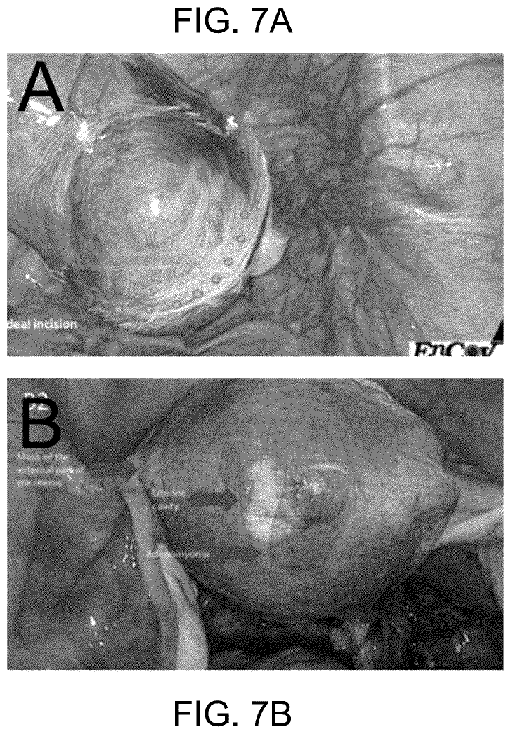

A study on the usefulness of mixed reality guidance for intra-operative procedures is discussed. FIGS. 7A and 7B show photos of augmented reality guidance systems. FIG. 7A shows an overlaid image of muscle fibers and spheres that suggest an ideal incision point to begin myomectomy. FIG. 7B shows an overlay of external wall of a uterus, uterine cavity, and location of adenomyoma to guide initial incision point, in accordance with various embodiments. In those cases, 3D renderings are generated from MRI scans and co-registration algorithms are used to overlay the renderings within the image from the laparoscope. However, these overlays only provide a 2D guidance since the location of the fibroid is only localized to the surface of the uterus. Furthermore, since the uterus of the location of the fibroids will change shape as they are removed, these co-registration methods cannot be used for cases with multiple fibroids. The disclosed systems and methods render the fibroids that are displayed separately from the laparoscopic image, showing a rendering of both the uterus and fibroids structures, such that their full 3D orientations can be intuitively understood. This overlay can allow for tracking of which fibroids are being removed to ensure nothing is left behind. This system can be helpful in a statistically significant manner.

An overview the disclosed aims are listed in Table 1. These aims are tailored to be conducted in parallel to ensure success without interdependence.

TABLE 1
Overview of Specific Aims

| | Deep Learning Models | | | | Input Data | | |
|---|---|---|---|---|---|---|---|
| | | | 3D CNN + | | 1500 | 1000 | |
| | | Hybrid | Tabular | | retrospective | retrospective | |
| | 3D CNN | Segmentation/ | Data | Recurrence | MRI scans + | MRI scans + | |
| Aim | Segmentation | Classification | Segmentation | Prognosis | tabular data | tabular data | Primary Outcome |
| 1 | X | X | X | | X | | Treatment Planning |
| 2 | | X | X | X | | X | Tumor Diagnosis |
| | | | | | | | Recurrence Prognosis |
| 3 | X | | X | | X | | Mixed Reality Guidance |
| | | | | | | | System |

A new dual-modality multitask deep learning model can be developed to jointly predict the prognosis (regression task) for the removal of the fibroids using minimally invasive procedure. To this end, both 3D volumetric MRI images and patients' metadata (structured tabular data) are leveraged to feed into the disclosed deep learning architecture. The model can be trained with retrospective images with ground truth classifications determined by procedure conversion to open surgery due to an inability to complete the procedure laparoscopically. This model's development can ensure that patients with uterine fibroids will be referred to the appropriate type of procedure to avoid aborted procedures and/or risk adverse outcomes.

The 1,500 MRI scans pertaining to patients with uterine fibroids will be divided into 3 parts: (i) training set (60%; 900 scans), (ii) validation set (20%; 300 scans), and (iii) testing set (20%; 300 scans). The training and validation set will be used during model training. The testing set will be used for model evaluation at the end of the model training. Additional patients can be obtained if further training or validation is needed. To ensure that both training and test dataset contain a fair representation of each class, datasets are randomly shuffled before splitting them into training and test sets. Additionally, before passing the inputs and ground truth in neural networks, data vectorization is applied to the datasets, turning MRI images and metadata into tensors of floating-point data. In various embodiments, extensive data augmentation including (the random shifting and scaling with a zoom range of 0.1 and the shift of 0.5 mm, random brightness and contrast changes, random flipping) is performed to avoid the overfitting training phase. The patient-level metadata comprise the pre-operative, Procedural, and post-operative attributes such as follows: i) pre-operative: Social History (age, BMI, surgical history, ADL, etc.), Socioeconomics (occupation, marital status, health maintenance-pap, vaccines, etc.), Imaging (IOTA score), and Blood Work (CA-125, HE4, ROMA test, OVA-1). ii) Procedural: Anesthesia ASA, Estimated Blood Loss, Total IV Fluids, Operative time, Year of resident, Robot used, Laparoscope used, Conversion rate, etc. iii) post-operative: Pathology, ovarian tumor size, Fibroid weight, ovarian tumor size.

A hybrid deep learning architecture that is highly efficient for simultaneous segmentation, classification, and prognosis of treatment planning concerning uterine fibroids is built in accordance with various embodiments disclosed herein. The deep learning model's implementation can be carried out on the PyTorch/Keras platform. The training and testing bed can include NVIDIA GTX 1080TI (B0-B4), Titan RTX (B5, B6) graphics card, and CUDA 9.0. In various embodiments, cloud services (Google Colab, AWS Deep Learning AMIs, Lambda GPU Cloud, and Azure) are utilized during pre-processing, post-processing, and prototyping. The overall steps in the developments of a deep learning model are as follows: i) Randomly initialize each model. ii) Train each model on the training set. iii) Evaluate each trained model's performance on the validation set. iv) Choose the model with the best validation set performance. v) Evaluate this chosen model on the test set. The detailed description regarding each step will be discussed in the following subsections.

To accurately segment the uterine fibroids, we're primarily leveraging the state-of-the-art Encoder-Decoder global convolutional network (called HIFUNet), which was published in late 2020. One of the significant shortcomings of this work is boundary inaccuracies in patients depicting multiple fibroids, which can often occur. The limitation is addressed and mitigated by employing 3D CNN where within HIFUNet, 2D convolutional and MaxPool layers are replaced with 3D convolutional filters and 3D MaxPooling layers using Keras. 1D CNN extracts spectral features, and 2D CNN extracts spatial features from the input data. Whereas 3D CNNs can consider both 1D and 2D CNNs by simultaneously extracting spectral and spatial features from the input volume. Consequently, 3D CNN features are advantageous in analyzing volumetric medical imaging, assuring the features learned by CNN are generalizable across raw datasets. Whether as an alternative or a comparison, MRI datasets are trained using Deep Medic. This famous work won the ISLES 2015 competition and became the state-of-the-art performance on 3D volumetric brain scans. Also, Deep Medic, a 3D CNN architecture, was carried forward by Kamnitsas et al. during the brain tumor segmentation (BRATS) 2016 challenge, where the authors took advantage of residual connections in 3D CNN. The results were remarkable and placed in the top 20 teams with median Dice scores of 0.898 (whole tumor, WT), 0.75 (tumor core, TC), and 0.72 (enhancing core, EC). Besides being a 3D CNN, another fact that makes the Deep Medic a rational architecture candidate to be tested on fibroids dataset is its capability in the face of variations in brain lesion size across different scans, causing imbalances in training samples. As mentioned above, the fibroids also are more difficult to segment than the uterus due to their unclear boundaries and undefined shapes. Yet, Deep Medic has always been implemented for brain tumor segmentation, and this would allow us to determine the robustness of the network in the presence of other organs using MRI scans. Both approaches will be comprehensively evaluated and compared to other deep learning methods (i.e., U-Net, HRNet, and CE-Net) using different quantitative measures. Additionally, the area-based indexes are used to compare the predicted segmentation results with the ground truth manually labeled by an expert. These indexes include the Dice coefficient (DSC), Precision, Sensitivity (SE), Specificity (SP), Jaccard index (JI), False Positive Ratio (FPR), False Negative Ratio (FNR) and False Region Ratio (FRR). The distance-based indexes are also used to evaluate the segmentation in terms of the location and shape accuracy of the extracted region boundaries, such as the Mean Absolute Distance (MAD, Maximum Distance (MAXD) and Hausdorff Distance (HD).

A robust deep learning model capable of selecting whether fibroids can be successfully removed using MI techniques or require open surgery is built. The facts that drive such a decision and selection of the correct treatment planning are imaging variables, such as the number of fibroids' exact location. For training the model, the existing correct decision that had been made previously for each MRI case is leveraged by designating two diagnostic categories, laparoscopy and laparotomy. A diagnostic class is assigned to each MRI case that is taken to be the ground truth. The network structures for the two tasks (segmentation and classification of uterine fibroids) are similar, and both aim to extract feature representations from the input MRI. Furthermore, training accurate DL models typically depend on large training sets; large numbers of queries may be required to build reliable DL models, which may incur a high annotation cost for each task at hand.

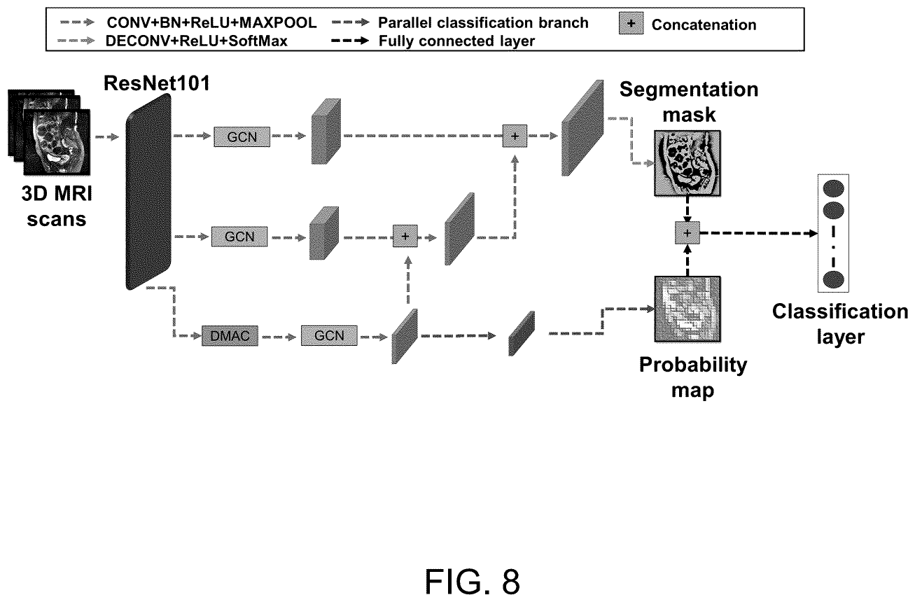

Hence, a framework is designed to train the two tasks jointly, as shown in FIG. 8. FIG. 8 illustrates a schematic of simultaneous segmentation and determination of the treatment strategy for uterine fibroids, in accordance with various embodiments. To this end, the Y-Net architecture (introduced in 2018 as a joint segmentation and classification network for the diagnosis of breast biopsy images) is leveraged. Y-Net outperformed the plain and residual encoder-decoder networks by 7% and 6%, respectively. The two ground truths per MRI case are i) a pixel-level annotations for segmentation, which already is available from the ground truth disclosed above, and ii) a class-level annotations (two diagnostic categories: laparoscopy and laparotomy). Y-Net allows two different outputs, an instance-level segmentation mask and an instance-level probability map. In various embodiments, the instance-based approach causes the segmentation accuracy to drop by about 1%, yet outperforming the state-of-the-art methods, 7% in accuracy. There are three contributions to Y-Net model as the followings: i) Evaluation of the Y-Net model to uterine fibroids and ovarian tumors, which is another applications of Y-Net other than for breast biopsy images. ii) Replace the instance-based approach with the semantic binary-based approach, anticipating the no-accuracy drop of the segmentation performance. iii) Replace the existing Y-Net's encoding blocks by the encoder module ResNet101 and the feature extractor GCN, which both are part of the HIFUNet as described above. Such expansion of the network not only allows taking advantage of GCN with deep multiple convolutions, which showed promising results, but also enables the model to jointly perform segmentation and classification. All three approaches are evaluated and compared to other deep learning models using the indexes described above. Additionally, the area under the curve (AUC), receiver operating characteristic (ROC) curve, and confusion matrix are used to measure the performance of a binary classifier.

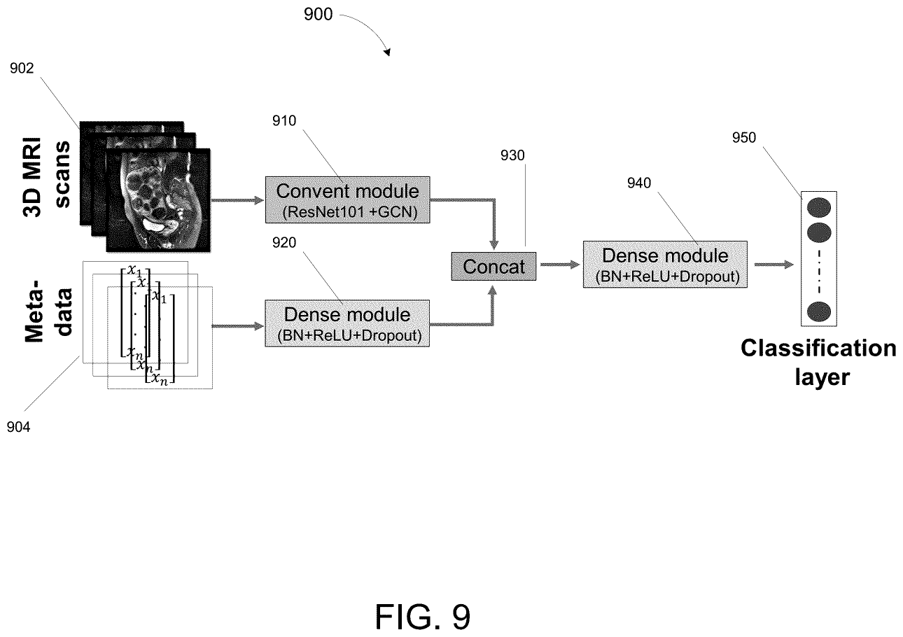

Dual-modality multitask deep learning model, leveraging both 3D volumetric MRI images 902 and patient-level metadata (structured tabular data) 904, as illustrated in FIG. 9 below. While imaging variables (i.e., shape, number, and location of fibroids) are rich information aiding the selection of the correct treatment planning for patients with uterine fibroids, the performance of the proposed deep learning model can be improved by incorporating both 3D volumetric MRI images and patient-level metadata (structured tabular data). To this end, convent module (containing the encoder module ResNet101 and the feature extractor GCN) are employed to be receptive to MRI images and implement a Dense module for the encoded patient-level metadata.

FIG. 9 illustrates an example schematic of a deep learning architecture 900 for dual-modality network, in accordance with various embodiments. The deep learning architecture for dual-modality network includes a Convent module 910, which includes Resnet101 backbone and GCN module, and a Dense module 920 to be receptive to patient-level metadata. The model's feature is concatenated in the Concat module 930 and is passed into Dense module 940 followed by the classification layer 950 (classifier). This can be followed by a late fusion technique to combine features. This is similar to the approach taken by the winners of the ISIC 2019 Skin Lesion Classification, where shown performance improvement by 1 to 2% through the incorporation of metadata. Initially, the CNNs are trained on MRI image data (task1). Then, the CNNs weights are frozen and the metadata neural network area attached. This time the metadata network's weights and the classification layer (task2) are trained. In order to pass the patient-level metadata 904 to a dense (fully-connected) neural network, the data is encoded as a feature vector where a one-hot encoding technique can be chosen. For evaluation, the mean sensitivity S is used for training with MRI images (task1) and then for training with metadata (task 2). Additionally, several metrics can be used to measure the classifier's performance (i.e., AUC, sensitivity, and specificity).

Development of a deep earning model to predict which patients are successful candidates for a minimally invasive procedure based on malignancy of an ovarian tumor is described. A new dual-modality multitask deep learning model can be developed to jointly classify a tumor as malignant or benign based on radiologic markers from a set of retrospective MRI scans. Ground truth classification can be determined by biologic tumor markers obtained post-procedurally. The development of this model can be the basis to automate the referral of patients to either a gynecologist or oncology gynecologist for the removal of ovarian tumors using laparoscopy.

The data will be prepared as described herein, except since there are 1,000 MRI scans pertaining to patients with ovarian tumors, the data can be divided into 3 parts: (i) training set (60%; 900 scans), (ii) validation set (20%; 300 scans), and (iii) testing set (20%; 300 scans).

The detailed description of the deep learning model's implementation can be the same as described above. Moreover, the steps taken in the development of the deep learning model can be applied to the ovarian tumor dataset, retraining the model for aims as described above.

Since the malignancy of an ovarian tumor dictates whether women should be referred to a gynecologist or gynecologic oncologist, the joint segmentation and classification on the ovarian tumor datasets are primarily executed. The following steps can be conducted to implement each of the successive DL models for the ovarian tumor datasets as follows: i) Feed 3D volumetric MRI into a 3D version of HIFUNet for multi-class segmentation of ovarian tumor, designating two diagnostic categories (benign or malignant) ii) Test Deep Medic, a 3D CNN architecture, for ovarian tumor segmentation. iii) Evaluation and comparison of the model in (i) and (ii) with other deep learning methods, as well as examine the predicted segmentation results with the ground-truth manually labeled by an expert using the indexes described above. iv) Implement the original Y-Net, for joint segmentation and classification of ovarian tumors. v) Test the modified version of Y-Net, described above, on ovarian tumors datasets. vi) Utilizing AUC, ROC curve, and confusion matrix to measure a binary classifier's performance.

Dual-modality multitask deep learning model includes leveraging both 3D volumetric MRI images and patient-level metadata (structured tabular data). The dual-modality multitask deep learning model, described herein, is employed to be tested on ovarian tumor datasets. The network architecture can remain the same, while the input dual-modality data can be changed according to the patients diagnosed with ovarian tumors. Hence, the model is retrained on these new datasets and evaluate the inference performance using the indexes described earlier.

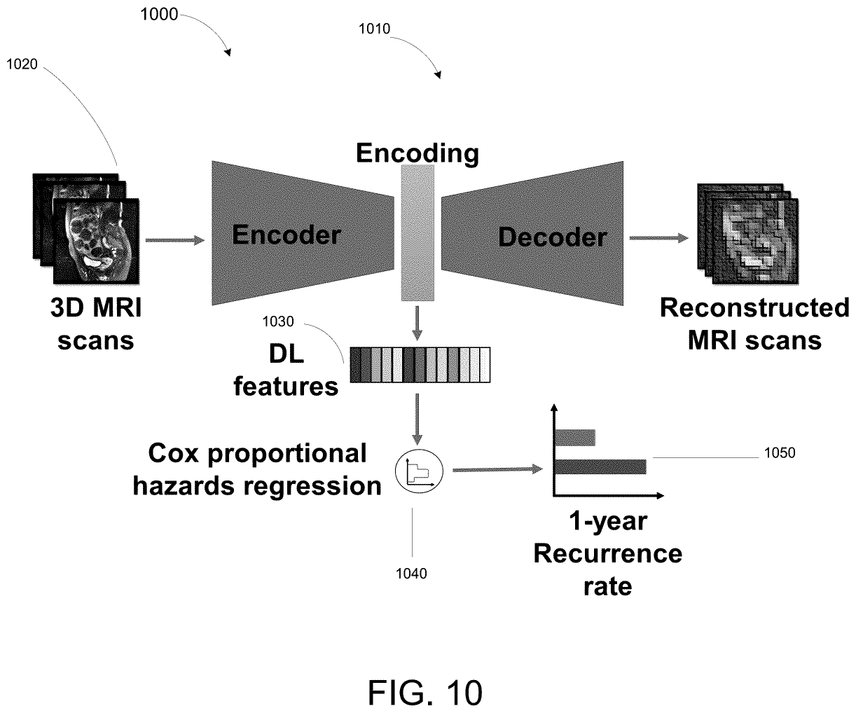

Prognosis the recurrence of ovarian tumors based on 1-year follow up with the patients is assessed. Recurrence is the leading risk for high-grade serious ovarian cancer (HGSOC) and accounts for 70-80% of ovarian cancer deaths, while overall survival has not changed significantly for several decades. There have been reports of several prognostic biomarkers associated with the recurrence of HGSOC, such as the stage of the International Federation of Gynecology and Obstetrics (FIGO) and preoperative serum cancer antigen (CA-125). Such clinical biomarkers are invasive and provide only limited information about tumors due to cancer's spatial and temporal pathologic heterogeneity. To this end, a semi-supervised learning self-training deep learning model can leverage MRI scans and metadata of both patients with and without follow-up information, as shown for example in FIG. 10.

FIG. 10 illustrates a schematic of the deep learning architecture to prognosis the recurrence of ovarian tumors, in accordance with various embodiments. The feature learning part is a convolutional autoencoder structure that encodes ovarian tumor into deep learning feature. The recurrent analysis part includes a multivariate Cox proportional hazard regression leveraging the imaging variables learned by the network to prognosis recurrence. To implement the proposed semi-supervised model 1000, an Encoder-Decoder global convolutional network 1010 (ResNet101 as the encoder module, GCN as the feature extractor, and U-Net concatenation operation as a decoder) is used to process imaging variables 1020 (e.g., 3D MRI scans), followed by multivariate Cox-PH regression 1040 to build the association between the deep learning features 1030 and the recurrence of HGSOC 1050 (shown as 1-year recurrence rate in FIG. 10). This overall recurrence analysis part can provide prognosis a hazard score indicating the individual recurrence risk 1050.

A deep learning model can be developed to automate the segmentation of uterine fibroids from a set of retrospective MRI scans. A mixed reality environment rendered from these patient-specific segmentations can enable physicians to visualize the relative positions of tumor fibroids for pre- and intra-procedural planning. The custom-software can allow voice-activated tracking of uterine fibroid removal in real-time. Intra-procedural guidance with and without using the mixed reality display can be compared in a pre-clinical study using a 3D printed model. The study can consist of physicians with and without experience performing laparoscopic procedures. The development of this guidance tool can potentially lower the learning curve for these minimally invasive procedures such that these lower risk procedures can be utilized for lower socio-economic populations.

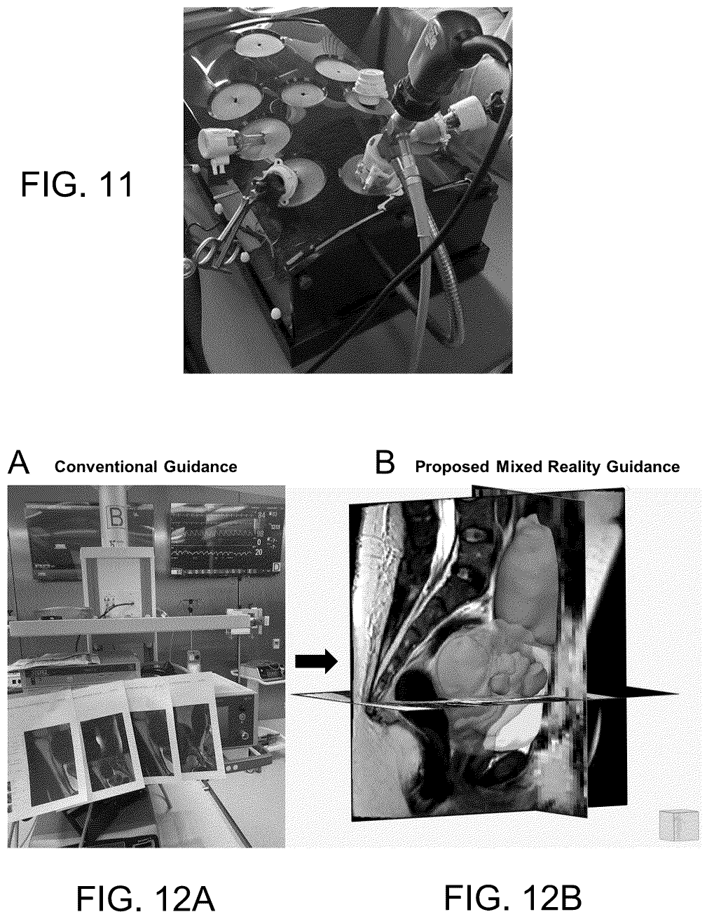

To create a training model that properly recapitulates laparoscopic removal of uterine fibroids, the following points are considered: i) Housing case that interfaces with standard laparoscopic tools. WCM has a state-of-the-art Skills Acquisition and Innovation Laboratory (SAIL) that has custom-made cases already able to perform this task. FIG. 11 shows a photo of Skills Acquisition and Innovation Laboratory (SAIL), which includes a laparoscopic trainer, in accordance with various embodiments. ii) Proper anatomic geometry. 10 phantom models are 3D printed with patient-specific uterine from MRI scans that provide a wide variety of number shapes, and locations of fibroids, and iii) Material properties. A digital multi-material 3D printer that uses the polyjet technology can be used to allow for material properties to be varied between mock tissue and fibroids. Phantom models that allow for tissue dissection and removal of mock fibroids will be optimized through iterative feedback. For each of these aspects, an expert can provide qualitative feedback to iterate upon until sufficient results are obtained based on the expert opinion. Evaluation can occur at the scheduled weekly. A training model can incur a wide variety of unintended events (e.g., puncture). To ensure these events are taking into account, mechanisms to visualize or alert their occurrence should be implemented, such adding food dyed fluid to mock arteries in the phantom model.

Deep learning models will be used for performing automated feature extraction to accomplish fibroid and uterine wall segmentation. HIFUNet, the state-of-the-art supervised deep learning architecture is used for the segmentation of various structures, such as the uterus, fibroids, endometrium, and bladder. The MRI images can be manually labelled by an expert, who is a trained radiologist. All ground truth annotations are double checked by an expert to ensure proper labelling is performed. This image set can serve as the ground truth for supervised CNN models. Similarly, a variant of U-Net architecture called 3D U-Net can be used to perform 3D segmentation to compare accuracy. The entire 1500 retrospective patient data set can be divided into 3 parts: (1) training set (60%; 1050 patients), (2) validation set (20%; 300 patients), and (3) testing set (20%; 300 patients). The training and validation set can be used during model training. The testing set can be used for model evaluation at the end of the model training. The performance of the model can be assessed on testing set by Dice coefficient. After the model segments the 3D geometry, the segmented features will be uploaded into the Unity software for visualization in mixed reality. This software will be uploaded onto Github for public distribution.

The mixed reality training can be designed to provide a 3D rendering of the fibroids so the user can have an intuitive interpretation for the location of the fibroids within the models. FIGS. 12A and 12B illustrate a concept of rendering mixed reality (MR) guidance display, in accordance with various embodiments. FIG. 12A shows an image depicting conventional method, where paper printouts of MRI cross-sections are taped in operating room. FIG. 12B shows a schematic of 3D rendering for MR guidance, where fibroids are shown within cross-sections of MRI cross-sections to give context of surrounding anatomy. The 3D rendering can be displayed within a Microsoft Hololens 2 mixed reality headset. The training software can be programmed to respond to voice-commands that allow for MRI cross-sections to scroll, anatomic features to toggle on and off, views to be rotated, and for individual fibroids to be selected as removed to keep track of the procedure. Surrounding anatomy, such as the uterine wall, endometrium, and bladder can be shown to provide orientation of the anatomy to the physician.

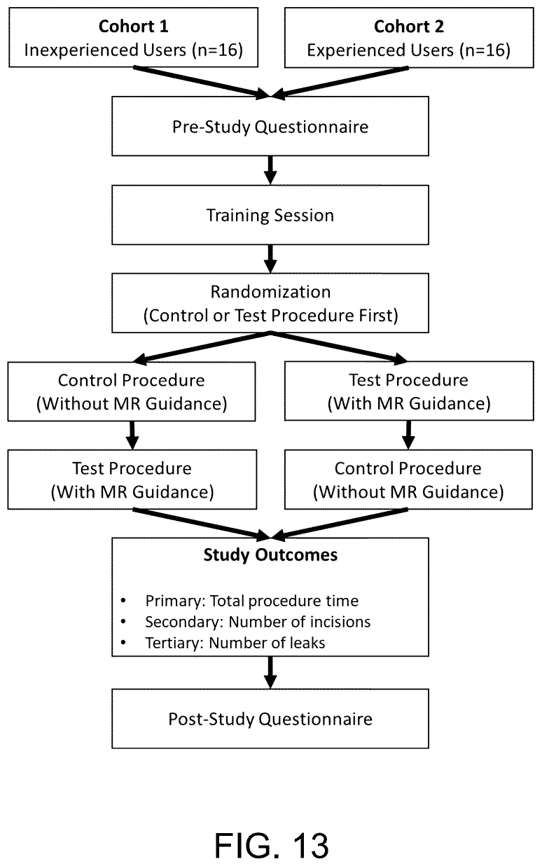

The potential impact of MR-guidance in laparoscopic procedures can be evaluated by comparing it to a control with no guidance during the removal of mock-fibroids in the 3D printed phantom model. A pilot study can be conducted with 2 cohorts. To illustrate this, FIG. 13 shows a schematic of study design to evaluate improved performance based on mixed reality (MR) guidance, in accordance with various embodiments. As illustrated in FIG. 13, the first of inexperienced users (i.e., fellows in training and attendings not credentialed to perform procedures), and the second of experienced users (attendings who have performed >20 procedure performed). An expert can lead the recruitment of both inexperienced participants, who can be recruited from WCM residency programs, and experienced participants, who will be recruited from both WCM and other centers in the NYC area. A pre-study questionnaire can confirm each participants experience. All participants can receive a training session to familiarize them with the equipment, procedure, and how to interface with the training model. Participants can perform the procedure both with and without guidance, but the order can be randomized to avoid bias from increased performance due to the additional experience. 2 types of models can be randomly assigned to either the control or test procedure also to avoid bias based on any inherent difference in ease of locating or removal of the mock-fibroids (the models can be designed to be of similar difficulty). In the control procedure, participants can be provided paper printouts of MRI scans and shown where the 10 fibroids are located. To increase the likelihood of obtaining data that differentiates these results, the 10 locations can be well distributed within the uterine model and of varying sizes and location within the uterine wall. Improved performance can be gauged by the primary study outcome of total duration of the procedure, and the secondary study outcome of the total number of incisions made into the model, as tracked by a study proctor. Suturing of the uterine wall after fibroid removal can be required and evaluated by the study proctor intra-procedurally to ensure that shortcuts are not taken to reduce procedure time. A post-study questionnaire can assess qualitative experience with the 3-D printed phantom and mixed reality guidance system.

All statistical analysis can be carried out using R Version 4.0.3 (R Core Team, Vienna, Austria). A paired t-test can be used to test the significance for the change in procedure time and number of incisions made by using the mixed reality guidance system. To explore the impact of participant experience, a mixed effects model can also be fit with a fixed effect for procedure, experience level, and the interaction of the two as well as a random effect for participant to account for correlation in the data. With a sample size of 16, at an alpha=0.05 level, using a one-sided paired t-test, there is 80% power to detect a medium effect size (0.65).

### RECITATION OF EMBODIMENTS

Embodiment 1. A method, system, computer-implemented method, and/or computer-based system for generating a model for performing gynecologic procedures, the method, system, computer-implemented method, and/or computer-based system comprising: a processor configured to execute machine-readable instructions borne by a non-transitory computer-readable memory device to cause the processor to: receive a first dataset comprising one or more gynecological tumor features; identify spectral and spatial features from the one or more gynecological tumor features from the first dataset; train a machine learning model using the identified spectral and spatial features, wherein the training comprises: performing a multi-class segmentation process based on the identified spectral and spatial features to produce a set of multi-class segmentation results, and classifying the identified spectral and spatial features by comparing the multi-class segmentation results with a ground-truth classification; validate the machine learning model using a second dataset; and optimize the machine learning model by modifying the machine learning model using a third dataset.

Embodiment 2: The method, system, computer-implemented method, and/or computer-based system of embodiment 1, wherein the first, second, and third datasets comprise a magnetic resonant imaging (MRI) dataset, a 3D MRI dataset, an ultrasound/sonogram dataset, a computed tomography (CT) dataset, or a doppler dataset, and subjects' metadata, and the spectral and spatial features include shapes and locations of the gynecological tumor features.

Embodiment 3: The method, system, computer-implemented method, and/or computer-based system of embodiments 1 or 2, wherein the ground-truth classification includes pixel-level annotations or class-level annotations.

Embodiment 4: The method, system, computer-implemented method, and/or computer-based system of any of embodiments 1-3, wherein performing the multi-class segmentation comprises: using area-based indexes to compare the multi-class segmentation results with the ground truth classification, or using distance-based indexes to further evaluate the multi-class segmentation in terms of location and shape accuracy of extracted region boundaries from the identified spectral and spatial features.

Embodiment 5: The method, system, computer-implemented method, and/or computer-based system of any of embodiments 1-4, wherein the first dataset comprises 3D magnetic resonant images (MRI) of uterine fibroids and the one or more gynecological tumor features comprise uterine fibroid features.

Embodiment 6: The method, system, computer-implemented method, and/or computer-based system of any of embodiments 1-5, wherein the first dataset comprises 3D magnetic resonant images (MRI) of ovarian tumors and the one or more gynecological tumor features comprise ovarian cancer features.

Embodiment 7: The method, system, computer-implemented method, and/or computer-based system of any of embodiments 1-6, wherein the machine learning model comprises a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

Embodiment 8: A method, system, computer-implemented method, and/or computer-based system of determining a success rate of a minimally invasive procedure for a patient, the method, system, computer-implemented method, and/or computer-based system comprising: a processor configured to execute machine-readable instructions borne by a non-transitory computer-readable memory device to cause the processor to: receive an imaging dataset comprising one or more scans of an anatomical area of interest for a potential procedure; analyze the imaging dataset using a machine learning model, wherein the machine learning model is trained using a multi-class segmentation of uterine regions from a plurality of scans for a plurality of subjects; identify one or more uterine fibroid features from the imaging dataset based on the analysis; and classify the one or more fibroid features, individually and/or as one or more groups, based on one or more characteristics of the one or more fibroid features.

Embodiment 9: The method, system, computer-implemented method, and/or computer-based system of embodiment 8, wherein the training of the machine learning model comprises instructions to cause the processor, upon execution of the instructions, to: perform a multi-class segmentation process based on a plurality of uterine fibroid features identified in a training dataset to produce a set of multi-class segmentation results, and classify the plurality of uterine fibroid features by comparing the multi-class segmentation results with a ground-truth classification.

Embodiment 10: The method, system, computer-implemented method, and/or computer-based system of embodiment 9, wherein the training of the machine learning model comprises instructions to cause the processor, upon execution of the instructions, to: perform a multi-class segmentation process based on a plurality of uterine fibroid features identified in a training dataset to produce a set of multi-class segmentation results, and classify the plurality of uterine fibroid features by comparing the multi-class segmentation results with a ground-truth classification.

Embodiment 11: The method, system, computer-implemented method, and/or computer-based system of any of embodiments 8-10, wherein the one or more identified uterine fibroid features comprise a shape, a number of, and relative positioning of the one or more uterine fibroids in the anatomical area of interest.

Embodiment 12: The method, system, computer-implemented method, and/or computer-based system of any of embodiments 8-11, further comprising instructions to cause the processor, upon execution of the instructions, to: output, via an output device, one or more representations of the one or more characteristics of the one or more fibroid features, wherein the one or more characteristics of the one or more fibroid features comprises a success rate of one or more types of surgical intervention for the one or more fibroid features.

Embodiment 13: The method, system, computer-implemented method, and/or computer-based system of any of embodiments 8-11, wherein the one or more characteristics of the one or more fibroid features used in the act of classifying the one or more fibroid features comprises a fibroid shape, a fibroid size, a number of fibroids, a fibroid position relative to at least one anatomical structure, a fibroid position relative to a blood vessel, or a fibroid position relative to at least one other fibroid.

Embodiment 14: The method, system, computer-implemented method, and/or computer-based system of any of embodiments 8-13, further comprising instructions to cause the processor, upon execution of the instructions, to: output, via an output device, one or more representations of the one or more fibroid features, either in isolation or in combination with the one or more characteristics of the one or more fibroid features.

Embodiment 15: The method, system, computer-implemented method, and/or computer-based system of any of embodiments 8-14, wherein the machine learning model comprises a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

Embodiment 16: The method, system, computer-implemented method, and/or computer-based system of embodiment 15, wherein the deep learning model is a dual-modality multitask deep learning model trained using the plurality of 3D volumetric MRI scans and patient-level metadata, wherein the CNN is trained using the plurality of 3D volumetric MRI scans, and wherein the patient-level metadata is encoded as a feature vector.

Embodiment 17: A method, system, computer-implemented method, and/or computer-based system for enhancing a diagnosis of an ovarian tumor, the method, system, computer-implemented method, and/or computer-based system comprising executing on a processor the steps of: receiving an imaging dataset comprising one or more scans of the ovarian tumor; analyzing the imaging dataset using a machine learning model, wherein the machine learning model is trained using a deep learning classification and a segmentation of a plurality of scans containing benign and malignant ovarian tumors; identifying one or more ovarian tumor features from the imaging dataset based on the analysis; and determining malignancy of the ovarian tumor based on the one or more identified ovarian tumor features.

Embodiment 18: The method, system, computer-implemented method, and/or computer-based system of embodiment 17, wherein the training of the machine learning model comprises: performing the deep learning classification and segmentation based on a plurality of ovarian tumor features identified in a training dataset to produce a set of multi-class segmentation results, and classifying the plurality of ovarian tumor features by comparing the multi-class segmentation results with a ground-truth classification.

Embodiment 19: The method, system, computer-implemented method, and/or computer-based system of embodiment 18, wherein the ground-truth classification includes pixel-level annotations or class-level annotations.

Embodiment 20: The method, system, computer-implemented method, and/or computer-based system of any of embodiments 17-19, further comprising: outputting, via an output device, one or more representations of the one or more ovarian tumor features and/or or more representations of a success rate of one or more types of surgical intervention for the one or more ovarian tumor features.

Embodiment 21: The method, system, computer-implemented method, and/or computer-based system of any of embodiments 17-20, wherein the one or more identified ovarian tumor features comprise a shape, a size, a number of, and relative positioning of one or more ovarian tumors in the MRI scans.

Embodiment 22: The method, system, computer-implemented method, and/or computer-based system of any of embodiments 17-21, wherein the machine learning model comprises a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

Embodiment 23: The method, system, computer-implemented method, and/or computer-based system of embodiment 22, wherein the deep learning model is a dual-modality multitask deep learning model trained using the plurality of 3D volumetric MRI scans and patient-level metadata, wherein the HIFUNet is trained using multi-class segmentation of an ovarian tumor, designating two diagnostic categories as benign or malignant, and wherein the CNN is trained using an ovarian tumor segmentation.

Embodiment 24: A method, system, computer-implemented method, and/or computer-based system of providing a mixed reality guidance for performing gynecological procedures, the method, system, computer-implemented method, and/or computer-based system comprising: a processor configured to execute machine-readable instructions borne by a non-transitory computer-readable memory device to cause the processor to: receive an imaging dataset comprising scans of an anatomical area of interest; perform automated segmentation of the scans using a 3D segmentation model, wherein the 3D segmentation model is trained using a deep learning multi-class segmentation of uterine regions; extract segmentation results comprising one or more structures of the anatomical area of interest; generate a 3D rendering using the one or more structures extracted from the automated segmentation; and display, via an electronic device, superimposed images from the 3D rendering overlayed with one or more scans.

Embodiment 25: The method, system, computer-implemented method, and/or computer-based system of embodiment 24, further comprising instructions to cause the processor, upon execution of the instructions, to: superimpose the 3D rendering with one or more images of the scans.

Embodiment 26: The method, system, computer-implemented method, and/or computer-based system of embodiments 24 or 25, further comprising instructions to cause the processor, upon execution of the instructions, to: manipulate the displayed superimposed images via a voice command.

Embodiment 27: The method, system, computer-implemented method, and/or computer-based system of any of embodiments 24-26, further comprising instructions to cause the processor, upon execution of the instructions, to: scroll the displayed superimposed images via a voice command.

Embodiment 28: The method, system, computer-implemented method, and/or computer-based system of any of embodiments 24-27, further comprising instructions to cause the processor, upon execution of the instructions, to: remove a structure from the 3D rendering; and updating the displayed superimposed images, whereby the updated displayed superimposed images display images without the removed structure.

Embodiment 29: The method, system, computer-implemented method, and/or computer-based system of any of embodiments 24-28, further comprising instructions to cause the processor, upon execution of the instructions, to: track one or more remaining structures based on the updated displayed superimposed images.

Embodiment 30: The method, system, computer-implemented method, and/or computer-based system of any of embodiments 24-29, wherein the one or more structures of the anatomical area of interest comprise a uterus, a fibroid, a cervix, a endometrium, a bladder, or an ovary.

Embodiment 31: The method, system, computer-implemented method, and/or computer-based system of any of embodiments 24-30, wherein the electronic device comprises a display, a monitor, a mixed reality device, an artificial reality device, or a virtual reality device.

What is claimed is:

1. A computer-based system for generating a model for performing gynecologic procedures, the system comprising:
a processor configured to execute machine-readable instructions borne by a non-transitory computer-readable memory device to cause the processor to:
receive a first dataset comprising one or more gynecological tumor features;
identify spectral and spatial features from the one or more gynecological tumor features from the first dataset;
train a machine learning model using the identified spectral and spatial features, wherein the training comprises:
performing a multi-class segmentation process based on the identified spectral and spatial features to produce a set of multi-class segmentation results, and
classifying the identified spectral and spatial features by comparing the multi-class segmentation results with a ground-truth classification;
validate the machine learning model using a second dataset; and
optimize the machine learning model by modifying the machine learning model using a third dataset.

2. The computer-based system of claim 1, wherein the first, second, and third datasets comprise a magnetic resonant imaging (MRI) dataset, a 3D MRI dataset, an ultrasound/sonogram dataset, a computed tomography (CT) dataset, or a doppler dataset, and subjects' metadata, and the spectral and spatial features include shapes and locations of the gynecological tumor features.

3. The computer-based system of claim 1, wherein the ground-truth classification includes pixel-level annotations or class-level annotations.

4. The computer-based system of claim 1, wherein performing the multi-class segmentation comprises:
using area-based indexes to compare the multi-class segmentation results with the ground truth classification, or
using distance-based indexes to further evaluate the multi-class segmentation in terms of location and shape accuracy of extracted region boundaries from the identified spectral and spatial features.

5. The computer-based system of claim 1, wherein the first dataset comprises 3D magnetic resonant images (MRI) of uterine fibroids and the one or more gynecological tumor features comprise uterine fibroid features.

6. The computer-based system of claim 1, wherein the first dataset comprises 3D magnetic resonant images (MRI) of ovarian tumors and the one or more gynecological tumor features comprise ovarian cancer features.

7. The computer-based system of claim 1, wherein the machine learning model comprises a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net.

8. A computer-based system for determining a success rate of a minimally invasive procedure for a patient, the system comprising:
a processor configured to execute machine-readable instructions borne by a non-transitory computer-readable memory device to cause the processor to:
receive an imaging dataset comprising one or more scans of an anatomical area of interest for a potential procedure;
analyze the imaging dataset using a machine learning model, wherein the machine learning model is trained using a multi-class segmentation of uterine regions from a plurality of scans for a plurality of subjects;
identify one or more uterine fibroid features from the imaging dataset based on the analysis; and
classify the one or more fibroid features, individually and/or as one or more groups, based on one or more characteristics of the one or more fibroid features.

9. The computer-based system of claim 8, wherein the training of the machine learning model comprises instructions to cause the processor, upon execution of the instructions, to:
perform a multi-class segmentation process based on a plurality of uterine fibroid features identified in a training dataset to produce a set of multi-class segmentation results, and
classify the plurality of uterine fibroid features by comparing the multi-class segmentation results with a ground-truth classification.

10. The computer-based system of claim 8, wherein the one or more identified uterine fibroid features comprise a shape, a number of, and relative positioning of the one or more uterine fibroids in the anatomical area of interest.

11. The computer-based system of claim 8, further comprising instructions to cause the processor, upon execution of the instructions, to:
output, via an output device, one or more representations of the one or more characteristics of the one or more fibroid features, wherein the one or more characteristics of the one or more fibroid features comprises a success rate of one or more types of surgical intervention for the one or more fibroid features.

12. The computer-based system of claim 8, wherein the one or more characteristics of the one or more fibroid features used in the act of classifying the one or more fibroid features comprises a fibroid shape, a fibroid size, a number of fibroids, a fibroid position relative to at least one anatomical structure, a fibroid position relative to a blood vessel, or a fibroid position relative to at least one other fibroid.

13. The computer-based system of claim 8, further comprising instructions to cause the processor, upon execution of the instructions, to:
output, via an output device, one or more representations of the one or more fibroid features, either in isolation or in combination with the one or more characteristics of the one or more fibroid features.

14. The computer-based system of claim 8, wherein the machine learning model comprises a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net, and wherein the deep learning model is a dual-modality multitask deep learning model trained using a plurality of 3D volumetric MRI scans and patient-level metadata, wherein the CNN is trained using the plurality of 3D volumetric MRI scans, and wherein the patient-level metadata is encoded as a feature vector.

15. A computer-based method for enhancing a diagnosis of an ovarian tumor, the method comprising executing on a processor the steps of:
receiving an imaging dataset comprising one or more scans of the ovarian tumor;
analyzing the imaging dataset using a machine learning model, wherein the machine learning model is trained using a deep learning classification and a segmentation of a plurality of scans containing benign and malignant ovarian tumors;
identifying one or more ovarian tumor features from the imaging dataset based on the analysis; and
determining malignancy of the ovarian tumor based on the one or more identified ovarian tumor features.

16. The computer-based method of claim 15, wherein the training of the machine learning model comprises:
performing the deep learning classification and segmentation based on a plurality of ovarian tumor features identified in a training dataset to produce a set of multi-class segmentation results, and
classifying the plurality of ovarian tumor features by comparing the multi-class segmentation results with a ground-truth classification.

17. The computer-based method of claim 15, wherein the ground-truth classification includes pixel-level annotations or class-level annotations.

18. The computer-based method of claim 15, further comprising:
outputting, via an output device, one or more representations of the one or more ovarian tumor features and/or or more representations of a success rate of one or more types of surgical intervention for the one or more ovarian tumor features.

19. The computer-based method of claim 15, wherein the one or more identified ovarian tumor features comprise a shape, a size, a number of, and relative positioning of one or more ovarian tumors in the MRI scans.

20. The computer-based method of claim 15, wherein the machine learning model is a deep learning model comprising a neural network from a list of convolution neural network (CNN), Fully Convolutional Network (FCN), Global Convolutional Network (GCN) with Deep Multiple Atrous Convolutions (DMAC), Encoder-Decoder global convolutional network (HIFUNet), U-Net, HRNet, and CE-Net, and wherein the deep learning model is a dual-modality multitask deep learning model trained using the plurality of 3D volumetric MRI scans and patient-level metadata, wherein the HIFUNet is trained using multi-class segmentation of an ovarian tumor, designating two diagnostic categories as benign or malignant, and wherein the CNN is trained using an ovarian tumor segmentation.
